(19) United States
(12) Patent Application Publication
(10) Pub. No.: US 2008/0124404 A1
(43) Pub. Date: May 29, 2008

(54) Hypolipidemic and/or hypocholesteremic compounds obtainable from the goldenseal plant

(76) Inventors: Jingwen Liu, Mountain View, CA (US); Parveen Abidi, Palo Alto, CA (US); Wei Chen, Sunnyvale, CA (US); Fredric B. Kraemer, Palo Alto, CA (US)

Correspondence Address: Bozicevic, Field & Francis LLP;Stanford University Office of Technology Licensing, 1900 University Avenue, Suite 200, East Palo Alto, CA, 94303, US

(21) Appl. No.: 11/820,288

(22) Filed: Jun. 18, 2007

Related U.S. Application Data
(60) Provisional application No. 60/815,222, filed on Jun. 19, 2006.

(51) Int. Cl. A61K 36/71; A61K 31/4375; A61P 9/00

(52) U.S. Cl. 424/726; 514/280

(57) ABSTRACT

The invention features bioactive compounds obtainable from goldenseal and methods of use of such compounds in reducing lipid (at least one of total cholesterol, LDL-cholesterol, free fatty acids, or triglycerides) in a patient having or suspected of having hyperlipidemia.

### CROSS-REFERENCE TO RELATED APPLICATION

This application claims the benefit of U.S. Provisional Application No. 60/815,222, filed Jun. 19, 2006, which application is incorporated herein by reference in its entirety.

### GOVERNMENT RIGHTS

This invention was made with government support from the Department of Veterans Affairs, Office of Research and Development, Medical Research Service, grant no. LIU0001 and the National Center for Complementary and Alterative Medicine, grant no. 1RO1 AT002543-01A1. The United States Government has certain rights in this invention.

### FIELD OF THE INVENTION

The present invention relates to reducing plasma total cholesterol, LDL-cholesterol, free fatty acids, and triglycerides.

### BACKGROUND

Coronary heart disease (CHD) is the major cause of morbidity and mortality in the United States and other Western countries. High blood plasma cholesterol concentration is one of the major risk factors for vascular disease and coronary heart disease in humans. Elevated low density lipoprotein cholesterol (LDL-cholesterol or LDL-c) and total cholesterol (TC) are directly related to an increased risk of coronary heart disease. A deficiency of high density lipoprotein cholesterol (HDL-cholesterol or HDL-c) can also be a risk factor for developing these conditions. Several clinical trials support a protective role of HDL-cholesterol against atherosclerosis.

The major plasma lipids, including cholesterol and the triglycerides, do not circulate freely in solution in plasma, but are bound to proteins and transported as macromolecular complexes called lipoproteins. The major lipoprotein classes are chylomicrons, very low density lipoproteins (VLDL), low density lipoproteins (LDL), and high density lipoproteins (HDL).

The major lipids transported in the blood are triglycerides; between 70 g and 150 g enter and leave the plasma daily, compared with 1 g to 2 g of cholesterol or phospholipids. Chylomicrons, the largest lipoproteins, carry exogenous triglyceride from the intestine via the thoracic duct to the venous system. VLDL carries endogenous triglyceride primarily from the liver to the peripheral sites for storage or use. The same lipases that act on chylomicrons degrade endogenous triglyceride quickly in VLDL, giving rise to intermediate density lipoproteins (IDL) that are shorn of much of their triglyceride and surface apoproteins. Within 2 to 6 hours, this IDL is degraded further by removal of more triglyceride, giving rise to LDL, which in turn has a plasma half-life of 2 to 3 days. VLDL is, therefore, the main source of plasma LDL.

Hypercholesterolemia can result either from overproduction or defective clearance of VLDL or from increased conversion of VLDL to LDL. Reduced clearance may be a result of diminished numbers of or abnormal function of the LDL receptors, which can result from genetic or dietary causes. Genetically mediated abnormal LDL receptor function usually results from molecular defects in the protein structure of the receptors. In humans, more than 70% of LDL is removed from the circulation by LDL receptor (LDLR) mediated uptake in the liver.

Expression levels of the hepatic LDLR therefore have a profound effect in influencing plasma cholesterol levels. Hepatic LDLR expression is regulated predominately at the transcriptional level by intracellular cholesterol pools through a negative feedback mechanism. When dietary cholesterol (as a constituent of chylomicron remnants) reaches the liver, the resulting elevated levels of intracellular cholesterol (or a metabolite of cholesterol in the hepatocyte) suppress LDL-receptor synthesis at the level LDL gene transcription. A reduced number of receptors results in higher levels of plasma LDL and therefore of TC. Saturated fatty acids also increase plasma LDL and TC levels; the mechanism of action is related to a reduced activity of LDL receptors. In the USA, dietary cholesterol and saturated fatty acid intake are high and are thought to account for an average increase of up to 25 to 40 mg/dL (0.65 to 1.03 mmol/L) of LDL blood levels, enough to increase significantly the risk of coronary artery disease (CAD).

Regulation of liver LDLR expression has been considered a key mechanism by which therapeutic agents could interfere with the development of CHD and atherosclerosis. For example, statins are specific inhibitors of HMG CoA reductase (HMR), the rate-limiting enzyme in cellular cholesterol biosynthesis. Depletion of the regulatory cholesterol pool in the liver results in increased LDLR expression and enhanced uptake of LDL particles from the circulation. Since the development of the first HMR inhibitor (HMRI) lovastatin, statin therapy has become the therapy of choice for hypercholesterolemia.

Despite the success of statin-based therapy, there remains interest in identifying additional cholesterol-lowering drugs. Berberine (BBR), an alkaloid isolated from the Chinese herb Huanglian, has been identified as a novel upregulator of hepatic LDLR (Kong et al. Nature Medicine 10, 1344-1352 (2004); Abidi et al. Arterioscler Thromb Vase Biol 25, 2170-2176 (2005)). BBR strongly increases LDLR mRNA and protein expression by extending the half-life of LDLR mRNA without affecting gene transcription, a mechanism of action different from statins. A placebo-controlled clinical study conducted in China showed that oral administration of BBR in 32 hypercholesterolemic patients at a daily dose of 1 g for 3 months reduced plasma total cholesterol (TC) by 29%, triglyceride (TG) by 35%, and LDL-c by 25% without side effects (Kong et al., supra). BBR is an indigenous component of other members of the plant family Ranunculaceae such as goldenseal (Hydrastis Canadensis Z.) (Herbalist, American Herbal Pharmacopoeia and Therapeutic Compendium 1, 1-36(2001)). Goldenseal is among the top 15 herbal products currently on the U.S. market and has been used to treat a variety of illnesses such as digestive disorders, urinary tract infection, and upper respiratory inflammation. (Herbalist, supra). There remains a need for compounds that can act as cholesterol-lowering agents.

### LITERATURE

WO 2006/029577; WO 83/03970; U.S. Pat. No. 6,239,139;

Kong et al. (2004) Nature Med. 10:1344-1351 (Epub Nov. 7, 2004); Abidi et al. (2005) Arterioscler. Thromb. Vasc. Biol. 25:2170-2176 (Epub Aug. 11, 2005); Stermitz et al. (2000) Proc. Natl Acad Sci 97:1433-1437; Qin et al. (2006) Bioorganic & Medicinal Chemistry, 14: 25-32; Herbalist, R. U. Goldenseal root hydrastis canadensis: standards of analysis, quality control, and therapeutics. American Herbal Pharmacopoeia and Therapeutic Compendium 1, 1-36(2001); Hsieh, P C et al. Proc. Natl. Acad. Sci. USA 1998, 95: 6602-6606; Samosorn, S et al. Bioorganic & Medicinal Chemistry 2006, 14: 857-865; Das et al. (2001) Synthetic Communications 31:1815-1817; Natural Standard Research Collaboration, Medline Plus record “Goldenseal (Hydrastis Canadensis L.), Berberine (Sep. 1, 2005).

U.S. Pat. No. 5,470,852; U.S. Pat. No. 6,974,799; U.S. Pat. No. 6,645,510; U.S. Pat. No. 6,551,628; U.S. Pat. No. 6,512,949; U.S. Pat. No. 6,440,465; U.S. Pat. No. 6,395,772; U.S. Pat. No. 6,387,416; U.S. Pat. No. 6,291,483; U.S. Pat. No. 6,280,768; U.S. Pat. No. 6,264,995; U.S. Pat. No. 6,248,309; U.S. Pat. No. 6,245,781; U.S. Pat. No. 6,162,393; U.S. Pat. No. 6,113,909; U.S. Pat. No. 6,008,356; U.S. Pat. No. 5,939,050; U.S. Pat. No. 5,928,645; U.S. Pat. No. 5,876,728; U.S. Pat. No. 5,856,487; U.S. Pat. No. 5,589,182; U.S. Pat. No. 5,153,178; U.S. Pat. No. 4,761,417; U.S. Pat. No. 4,701,458; U.S. Pat. No. 4,200,629; U.S. Pat. No. 4,087,426; U.S. Pat. No. 3,943,251; US 20060110415; US 20050060028; US 20040097534; US 20040091556

### SUMMARY OF THE INVENTION

The invention features bioactive compounds obtainable from goldenseal and methods of use of such compounds in reducing lipid (at least one of total cholesterol, LDL-cholesterol, free fatty acids, or triglycerides) in a patient having or suspected of having hyperlipidemia,

In one aspect, the invention relates to methods of reducing serum lipid (at least one of total cholesterol, LDL-cholesterol, free fatty acids, or triglycerides) in a patient having or suspected of having hyperlipidemia, which includes administering to said patient an effective amount of substantially pure canadine or a pharmaceutically acceptable salt thereof.

In another aspect, the invention relates to methods of reducing serum lipid (at least one of total cholesterol, LDL-cholesterol, free fatty acids or triglycerides) in a patient having or suspected of having hyperlipidemia, which includes administering to said patient an effective amount of one or more substantially pure hypolipidemic and/or hypocholesteremic compounds isolated from the goldenseal plant or pharmaceutically acceptable salts thereof, with the proviso that the compound isolated is not berberine. In a related embodiment, the invention provides substantially pure hypolipidemic and/or hypocholesteremic compounds obtained from goldenseal root extract.

In another aspect, the invention features methods of reducing serum lipid (at least one of total cholesterol, LDL-cholesterol, free fatty acids, or triglycerides) in a patient having or suspected of having hyperlipidemia, which includes administering to said patient an effective amount of a composition comprising berberine or a pharmaceutically acceptable salt thereof and a multi-drug resistant pump (MDR) inhibitor or a pharmaceutically acceptable salt thereof.

In related embodiments to the above, the invention relates to methods of increasing the ratio of HDL-cholesterol:LDL-cholesterol in a patient in need thereof, which includes administering to said patient an effective amount of substantially pure canadine or a pharmaceutically acceptable salt thereof.

In further related embodiments, the invention relates to methods of raising the ration of HDL-cholesterol:LDL-cholesterol in a patient in need thereof, which includes administering to said patient an effective amount of one or more substantially pure hypolipidemic and/or hypocholesteremic compounds isolated from the goldenseal plant or pharmaceutically acceptable salts thereof, with the proviso that the compound isolated is not berberine. In a related embodiment, the substantially pure hypolipidemic and/or hypocholesteremic compounds are isolated from goldenseal root extract.

In further related embodiments, the invention relates to methods of raising the HDL-cholesterol:LDL-cholesterol ratio in a patient in need thereof, which includes administering to said patient an effective amount of a composition comprising berberine or a pharmaceutically acceptable salt thereof and a MDR inhibitor or a pharmaceutically acceptable salt thereof.

In further embodiments, the invention relates to methods of treating a patient for a medical condition in which lowering of at least one of total cholesterol, LDL-cholesterol, free fatty acids, or triglycerides is beneficial, which includes administering to said patient in need of such treatment an effective amount of substantially pure canadine.

In still further embodiments, the invention relates to methods of treating a patient for a medical condition in which lowering serum lipid (at least one of total cholesterol, LDL-cholesterol, free fatty acids, or triglycerides) is beneficial, which includes administering to said patient in need of such treatment an effective amount of one or more substantially pure hypolipidemic and/or hypocholesteremic compounds isolated from the goldenseal plant or pharmaceutically acceptable salts thereof, with the proviso that the compound isolated is not berberine. In related embodiments, the substantially pure hypolipidemic and/or hypocholesteremic compounds are isolated from goldenseal root extract.

In other embodiments, the invention relates to methods of treating a patient for a medical condition in which lowering serum lipid (at least one of total cholesterol, LDL-cholesterol, free fatty acids, or triglycerides) is beneficial, which includes administering to said patient an effective amount of a composition comprising berberine or a pharmaceutically acceptable salt thereof and a MDR inhibitor or a pharmaceutically acceptable salt thereof.

In further embodiments, the invention relates to methods of reducing serum lipid (at least one of total cholesterol, LDL-cholesterol, free fatty acids, or triglycerides) in a patient having or suspected of having hyperlipidemia, wherein the method includes administering to said patient an effective amount of a formulation comprising at least one of the following agents or a pharmaceutically acceptable salt thereof:
substantially pure canadine,
Factor F3, wherein Factor F3 is produced by isolating from the goldenseal plant by preparative HPLC,
Factor F6, wherein Factor F6 is produced by isolating from the goldenseal plant by preparative HPLC, or
berberine in combination with an MDR inhibitor.
   wherein said administering is effective to reduce at least one of total cholesterol, LDL-cholesterol, free fatty acids, or triglycerides in said patient.

In further embodiments, the invention relates to methods for preventing or treating hyperlipidemia in a patient in need of such prevention or treatment wherein the method includes administering an anti-hyperlipidemia effective amount of substantially pure canadine or a pharmaceutically acceptable salt, isomer, or enantiomer thereof.

In further embodiments, the invention relates to methods for preventing or treating one or more symptoms of a cardiovascular disease or condition caused by hyperlipidemia in a patient in need of such prevention or treatment wherein the method includes administering an anti-hyperlipidemic effective amount of substantially pure canadine or a pharmaceutically acceptable salt, isomer, or enantiomer thereof.

In further embodiments, the invention relates to methods of controlling hyperlipidemia in a patient to reduce or prevent cardiovascular disease wherein the method includes administering to said patient an anti-hyperlipidemia effective amount of substantially pure canadine or a pharmaceutically acceptable salt, isomer, or enantiomer thereof.

In further embodiments, the invention relates to methods for treating one or more symptoms of cardiovascular disease wherein the method includes administering to a patient in need of such treatment an effective amount of substantially pure canadine or a pharmaceutically acceptable salt, isomer, or enantiomer thereof.

In further embodiments, the invention relates to methods of modulating LDLR expression in a patient wherein the method includes administering to a patient in need of such treatment an effective amount of substantially pure canadine or a pharmaceutically acceptable salt, isomer, or enantiomer thereof.

In a specific embodiment, the invention relates to methods of modulating LDLR expression in a patient wherein the method includes administering to a patient in need of such treatment an effective amount of substantially pure canadine or a pharmaceutically acceptable salt, isomer, or enantiomer thereof, wherein the substantially pure canadine is administered in combination with at least one anti-hyperlipidemic agent or adjunctive therapeutic agent useful in the treatment of cardiovascular disease.

In further embodiments, the invention relates to methods for increasing LDLR mRNA stability in a mammalian cell, tissue, organ, or patient wherein the method includes administering to said mammalian cell, tissue, organ, or patient in need of such increasing an effective amount of substantially pure canadine or a pharmaceutically acceptable salt, isomer, or enantiomer thereof.

In further embodiments, the invention relates to methods for modulating ERK activation in a mammalian cell, tissue, organ, or patient wherein the method includes administering to said mammalian cell, tissue, organ, or patient in need of such modulating of ERK activation an ERK activation modulatory effective amount of substantially pure canadine or a pharmaceutically acceptable salt, isomer, or enantiomer thereof.

In further embodiments, the invention relates to methods of lowering cholesterol in a mammalian cell, tissue, organ, or patient wherein the method includes administering to said mammalian cell, tissue, organ, or patient in need of such lowering a cholesterol lowering effective amount of substantially pure canadine or a pharmaceutically acceptable salt, isomer, or enantiomer thereof.

In another aspect, the invention relates to a pharmaceutical composition in unit dosage form including berberine or a pharmaceutically acceptable salt thereof and an MDR1 multidrug pump inhibitor or a pharmaceutically acceptable salt thereof and a pharmaceutically acceptable excipient.

In another aspect, the invention relates to a kit including unit doses in separate containers of berberine or a pharmaceutically acceptable salt thereof and an MDR1 multidrug pump inhibitor or a pharmaceutically acceptable salt thereof and an informational and/or instructional package insert.

In another aspect, the invention relates to a pharmaceutical composition including a mixture of berberine or a pharmaceutically acceptable salt thereof and an MDR1 multidrug pump inhibitor or a pharmaceutically acceptable salt thereof and a pharmaceutically acceptable excipient.

In another aspect, the invention relates to a pharmaceutical composition including berberine or a pharmaceutically acceptable salt thereof and a pharmaceutically acceptable excipient administered in combination with an MDR1 multidrug pump inhibitor or a pharmaceutically acceptable salt thereof and a pharmaceutically acceptable excipient.

In another aspect, the invention relates to a pharmaceutical composition comprising Factor F3, wherein Factor F3 is produced by isolation from the goldenseal plant by preparative HPLC, and a pharmaceutically acceptable excipient.

In another aspect, the invention relates to a pharmaceutical composition comprising Factor F6, wherein Factor F6 is produced by isolation from the goldenseal plant by preparative HPLC, and a pharmaceutically acceptable excipient.

In another aspect, the invention relates to a pharmaceutical composition for preventing or alleviating hyperlipidemia in a patient including an anti-hyperlipidemia effective amount of substantially pure canadine or a pharmaceutically acceptable sal, isomer, or enantiomer t thereof, and a pharmaceutically acceptable excipient.

In another aspect, the invention relates to a pharmaceutical composition for treating or preventing hyperlipidemia in a patient including an anti-hyperlipidemia effective amount of substantially pure canadine or a pharmaceutically acceptable salt, isomer, or enantiomer thereof, in combination with at least one anti-hyperlipidemic agent or adjunctive therapeutic agent useful in the treatment of cardiovascular disease.

In another aspect, the invention relates to a pharmaceutical composition for increasing LDLR expression in a mammalian cell, tissue, organ, or patient, including an LDLR effective amount of substantially pure canadine or a pharmaceutically acceptable salt, isomer, or enantiomer thereof, and a pharmaceutically acceptable excipient.

In another aspect, the invention relates to a pharmaceutical composition for increasing LDLR mRNA stability in a mammalian cell, tissue, organ, or patient, including an LDLR mRNA stabilizing amount of substantially pure canadine or a pharmaceutically acceptable salt, isomer, or enantiomer thereof, and a pharmaceutically acceptable excipient.

### BRIEF DESCRIPTION OF THE DRAWINGS

FIG. 1A-1F illustrates the upregulation of LDLR expression by goldenseal, CND, and BBR in HepG2 cells:

FIG. 1A: Chemical structures of berberine (BBR), canadine (CND), palmatine (PMT), β-hydrastine (HDT), and hydrastinine (HDTN).

FIG. 1B: Northern blot analysis of LDLR mRNA expression: HepG2 cells cultured in EMEM containing 0.5% FBS were treated with each compound at a dose of 20 μg/ml or with goldenseal (GS) from different suppliers at a dose of 2.5 μl/ml for 8 h. Total RNA was isolated and 15 μg per sample was analyzed for LDLR mRNA by northern blot. The membrane was stripped and hybridized to a human GAPDH probe. The figure shown is representative of 3 separate studies.

FIG. 1C: Real-time quantitative RT-PCR analysis: Effects of goldenseal and each alkaloid on LDLR mRNA expression in HepG2 cells were independently examined with quantitative real-time PCR assays. LDLR mRNA levels were corrected by measuring GAPDH mRNA levels. The abundance of LDLR mRNA in untreated cells was defined as 1, and the amounts of LDLR mRNA from drug-treated cells were plotted relative to that value. The figure is representative of 3-5 independent assays.

FIG. 1D: Dil-LDL uptake: HepG2 cells were treated for 18 h with 10 μg/ml BBR or with1.5 μ/ml goldenseal (equivalent to 10.2 μg/ml BBR). Thereafter, Dil-LDL was added to medium at a final concentration of 6 μg/ml and cells were trypsinized 4 h later. The uptake of Dil-LDL was measured by FACScan with 2×10⁴ cells per sample. The mean fluorescence value (MFV) of untreated cells is expressed as 100%. The data shown are representative of 2 separate assays.

FIG. 1E: Analysis of LDLR promoter activity: HepG2 cells were cotransfected with pLDLR234Luc and a normalizing vector pRL-SV40. After an overnight incubation, GW707 (2 μM), OM (50 ng/ml), BBR (15 μg/ml), CND (15 μg/ml), goldenseal (2.2 μ/ml), F3 (3 μ/ml), and F6 (3 μ/ml) were added to cells for 8 h prior to cell lysis. Firefly luciferase and renilla luciferase activities were measured. The data represent normalized LDLR promoter activity.

FIG. 1F: Regulation of LDLR mRNA stability by goldenseal: HepG2 cells were untreated or treated with actinomycin D at a dose of (5 μg/ml) for 30 min prior to the addition of BBR (15 μg/ml), CND (15 μg/ml), or goldenseal (2.2 μl/ml). Total RNA was harvested after 4 h and expression levels of LDLR mRNA were determined by real-time quantitative RT-PCR. The abundance of LDLR mRNA in cells cultured without actinomycin D was defined as 1, and the amounts of LDLR mRNA from actinomycin D-treated cells without or with herbal drugs were plotted relative to that value.

FIG. 2 illustrates the comparison of dose-dependent effects of CND and BBR on LDLR mRNA expression. HepG2 cells were treated with CND or BBR for 8 h at the indicated concentrations and total RNA was isolated for analysis of LDLR mRNA and GAPDH mRNA expression by northern blot (FIG. 2, Panel A) and real-time PCR assays (FIG. 2, Panel B).

FIG. 3 illustrates the separation of goldenseal extract by silica gel column and detection of LDLR modulation activity in column eluates. In FIG. 3, Panel A, 1 ml goldenseal extract was separated into 26 fractions by silica gel column using chloroform/methanol as the elution solvent. The fluorescent intensity of 200 μl from each fraction was measured by a fluorescent microplate reader at 350-nm excitation and 545-nm emission. The presence of CND, HDT, or BBR in eluates were determined by HPLC and LC-MS with standard solutions of each compound as the reference. In FIG. 3, Panel B, HepG2 cells were treated for 8 h with 1.5 or 3 μl of each fraction after evaporation of the solvent and redissolving in DMSO. BBR (15 μg/ml) and goldenseal (2.2 μl/ml) were used in these experiments as positive controls. The inducing effects of F3 and F6 on LDLR mRNA expression were consistently observed in 4 separate experiments.

FIG. 4 illustrates the kinetic studies of LDLR expression and uptake of BBR in HepG2 cells:

(FIG. , Panel A) Time-dependent inductions of LDLR mRNA expression by goldenseal and BBR: HepG2 cells were incubated with BBR (15 μg/ml) or goldenseal (2.2 μl/ml) for the indicated times. The abundance of LDLR mRNA was determined by quantitative real-time PCR assays.

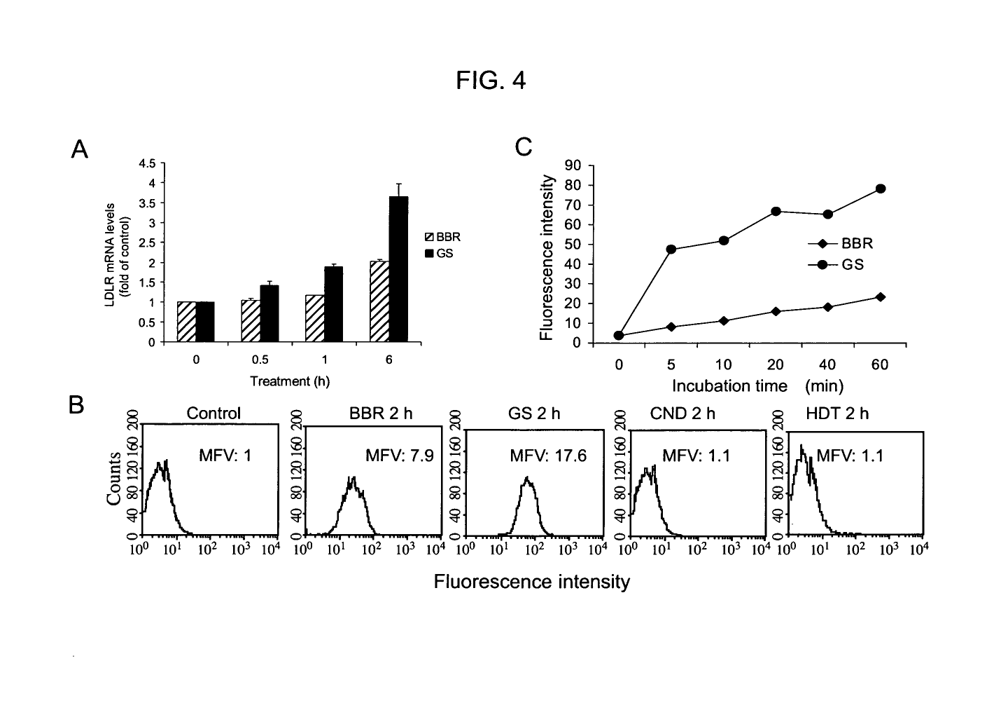

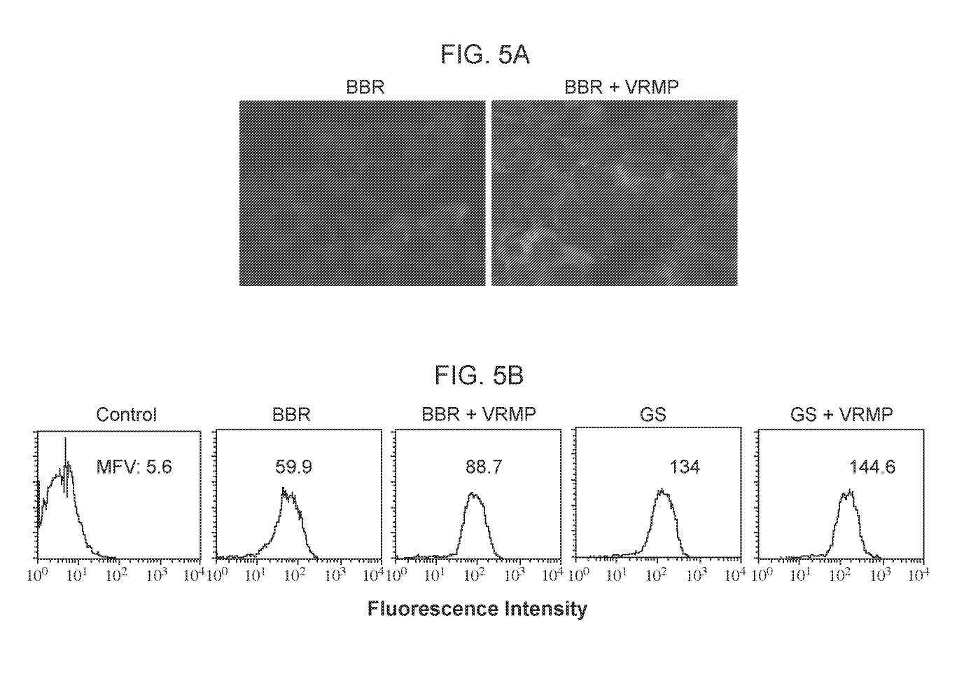

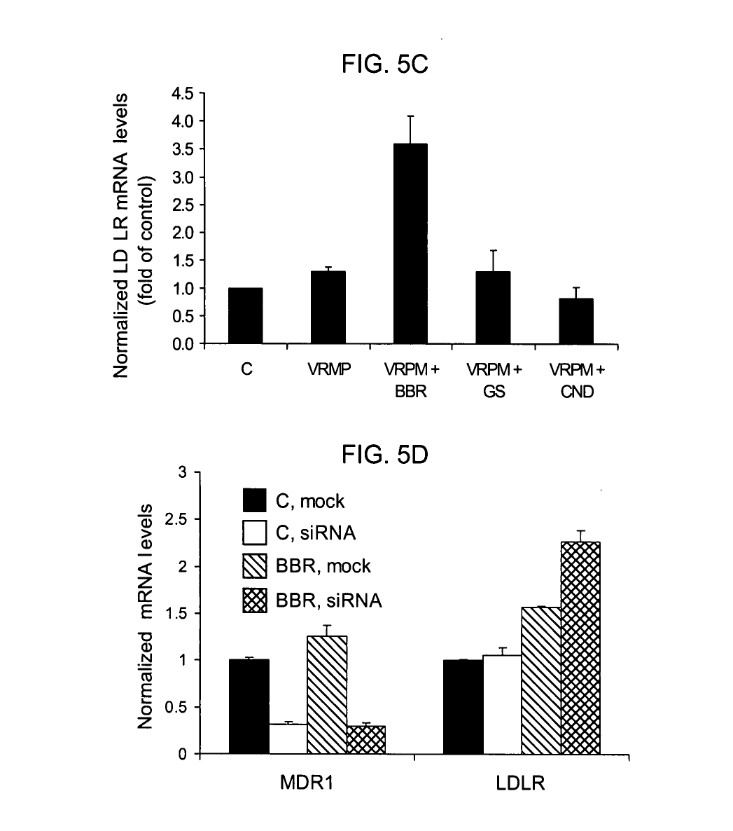

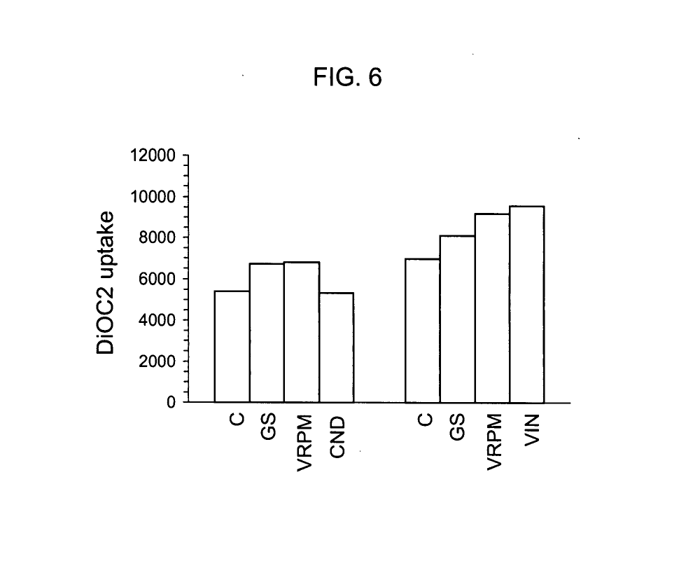

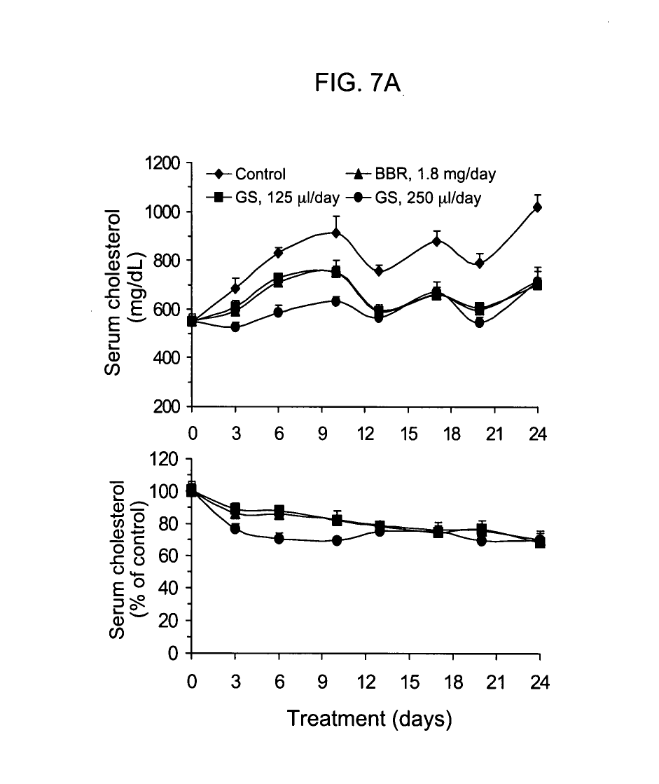

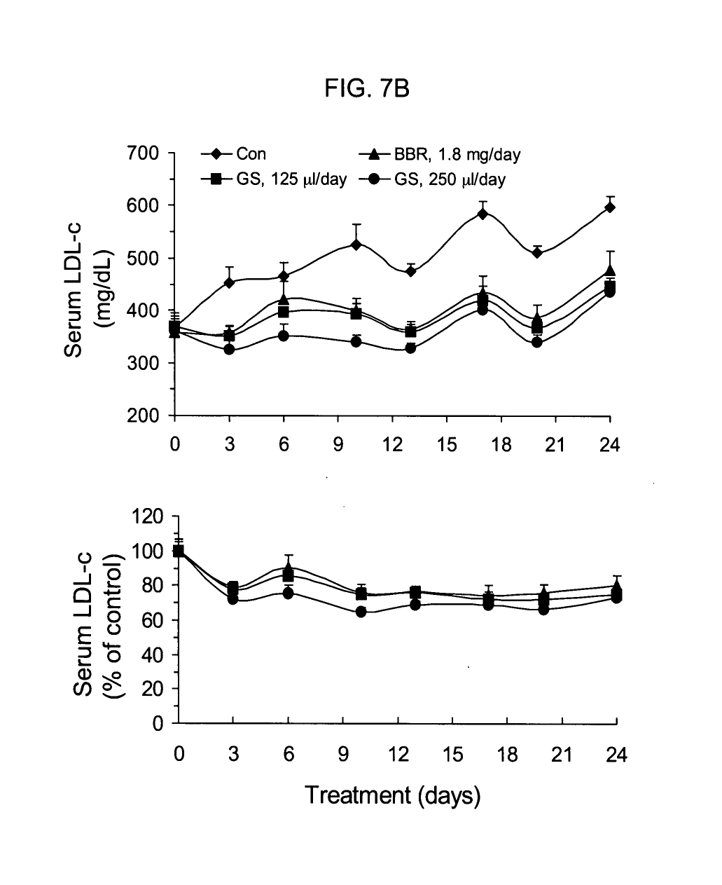

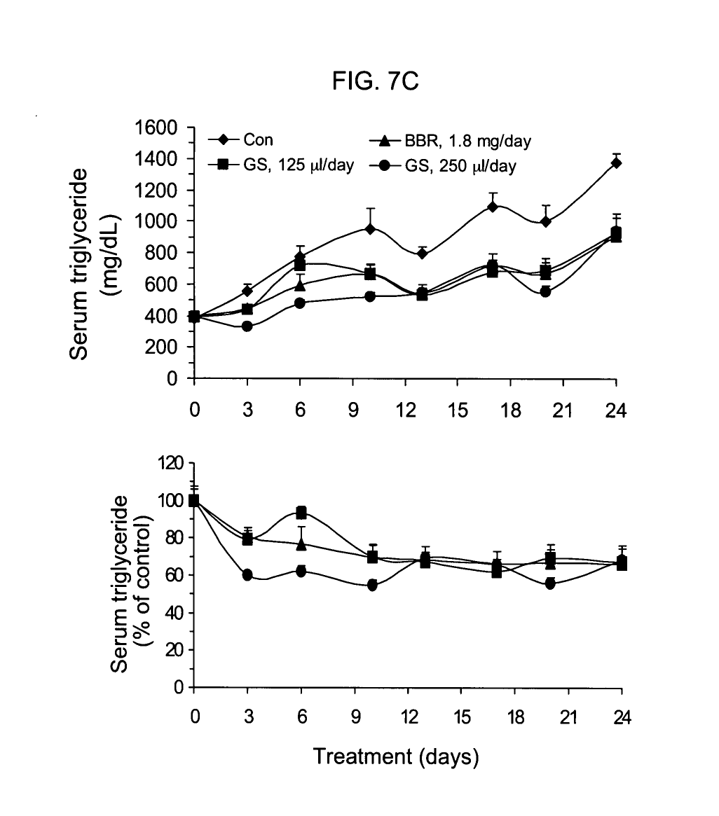

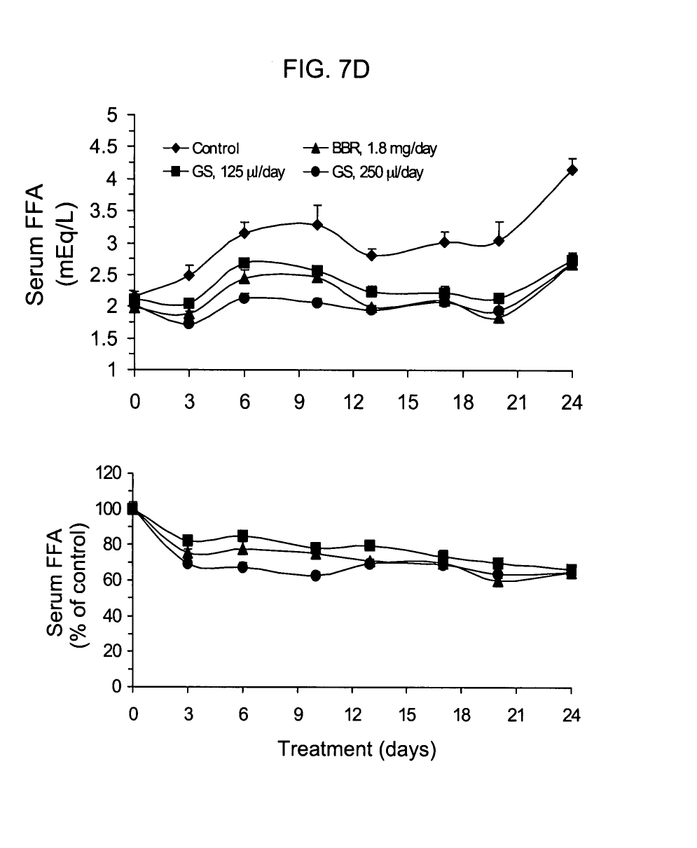

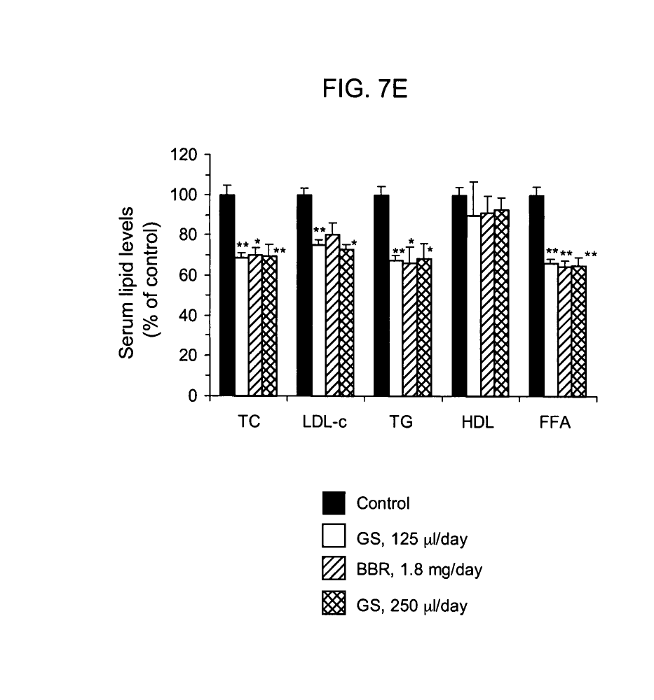

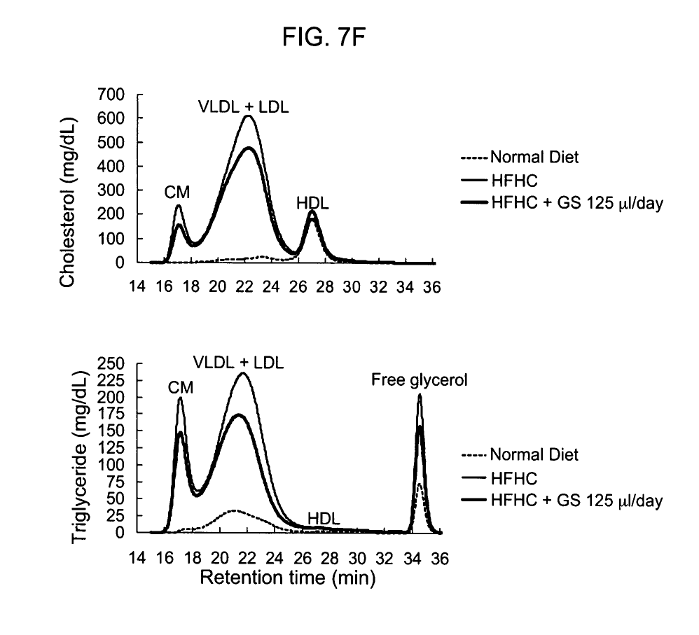

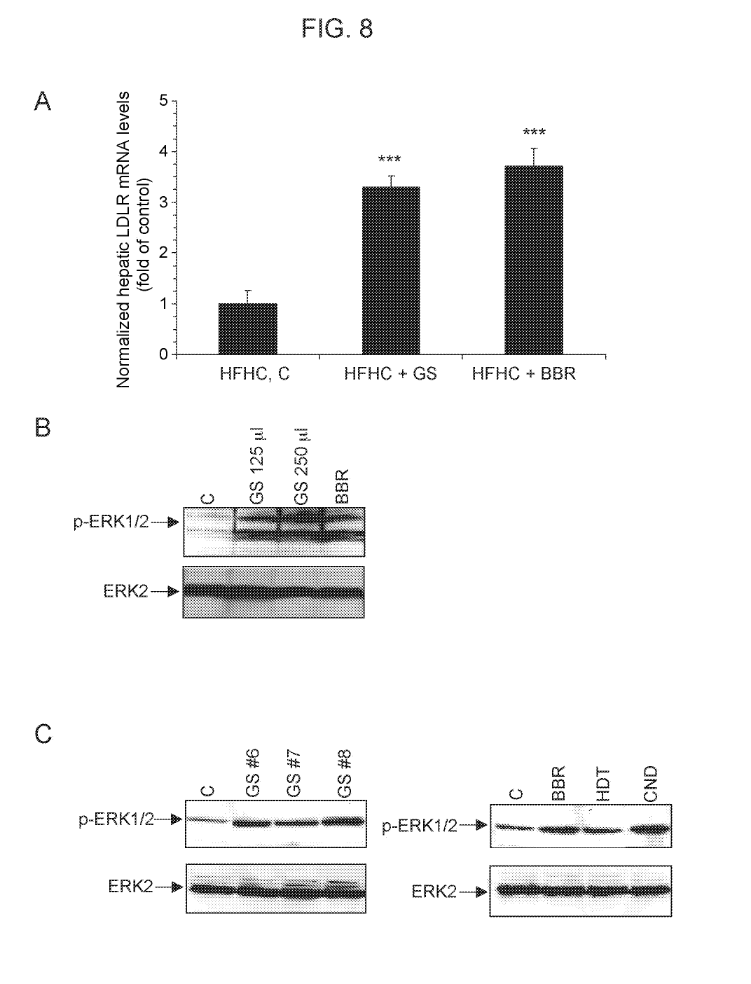

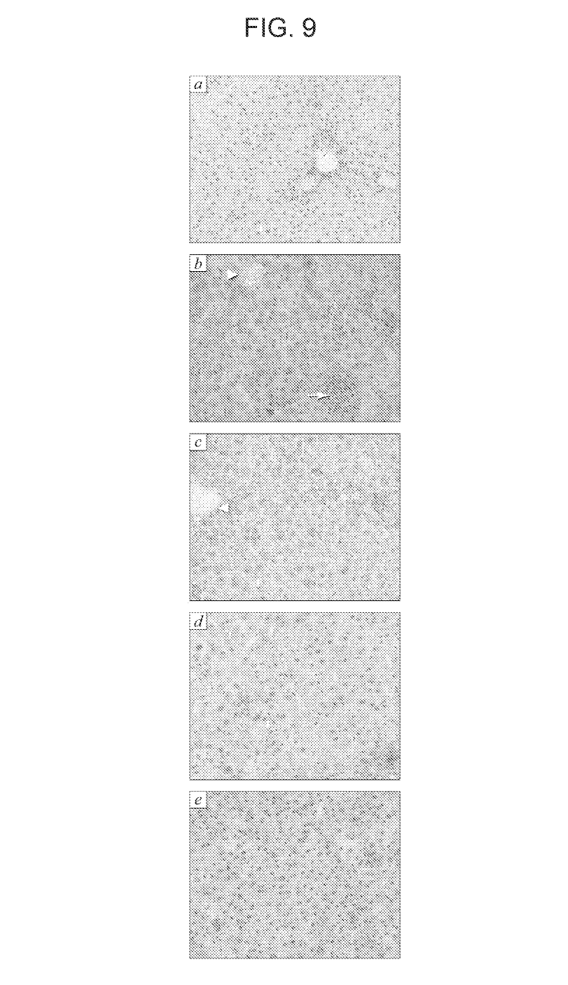

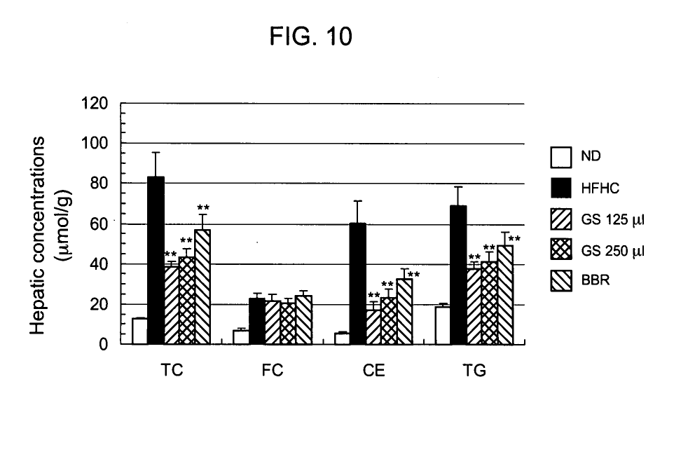

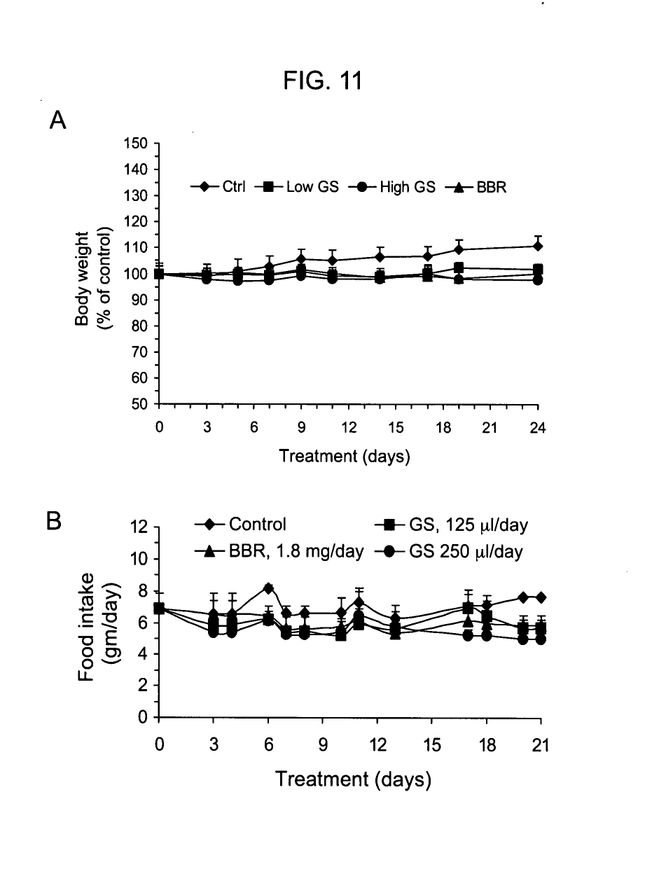

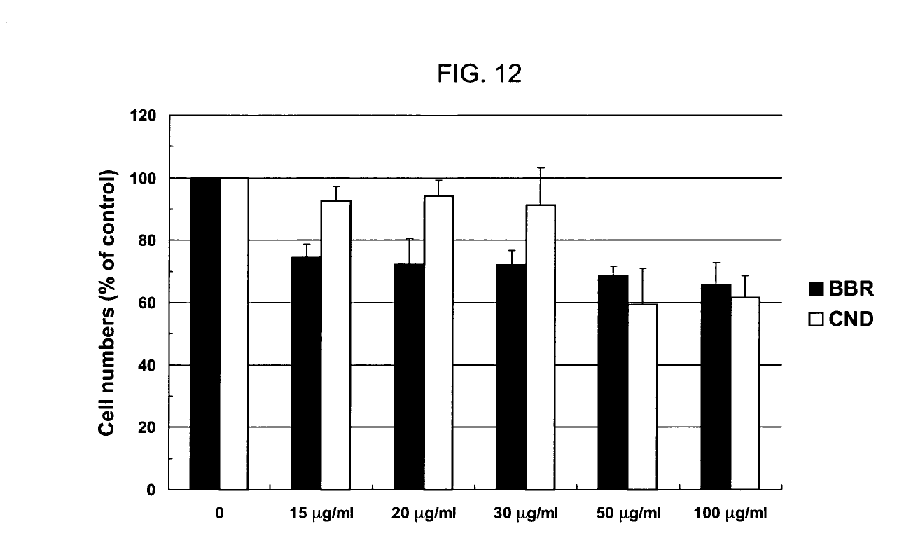

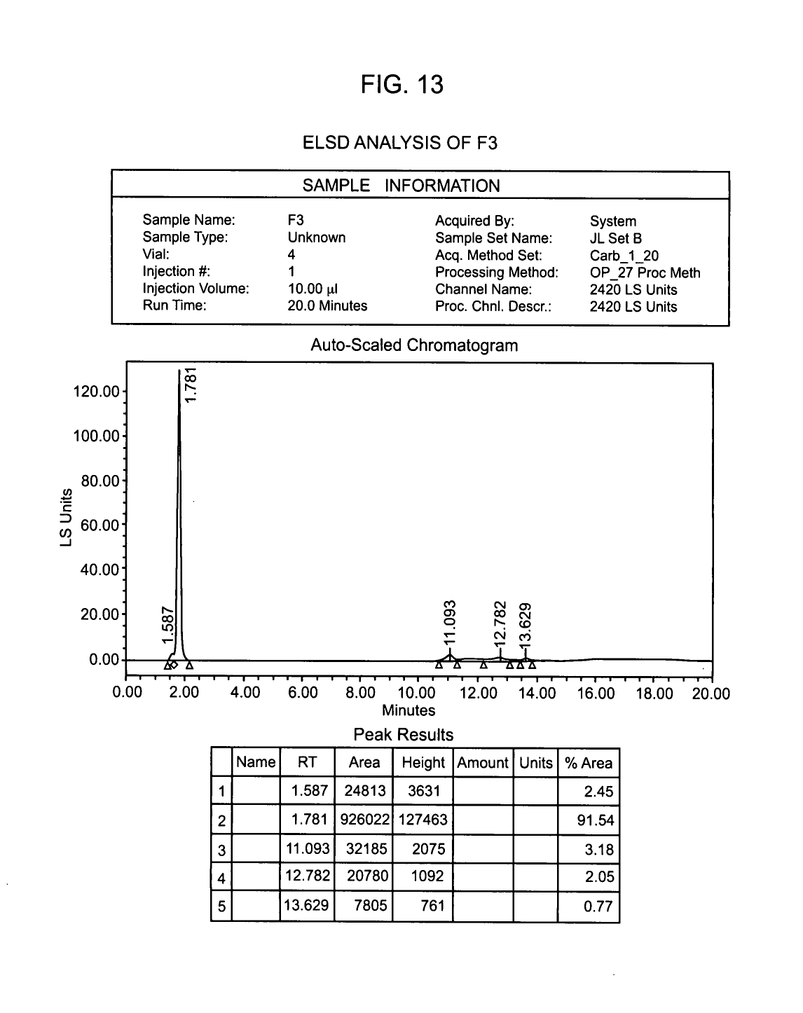

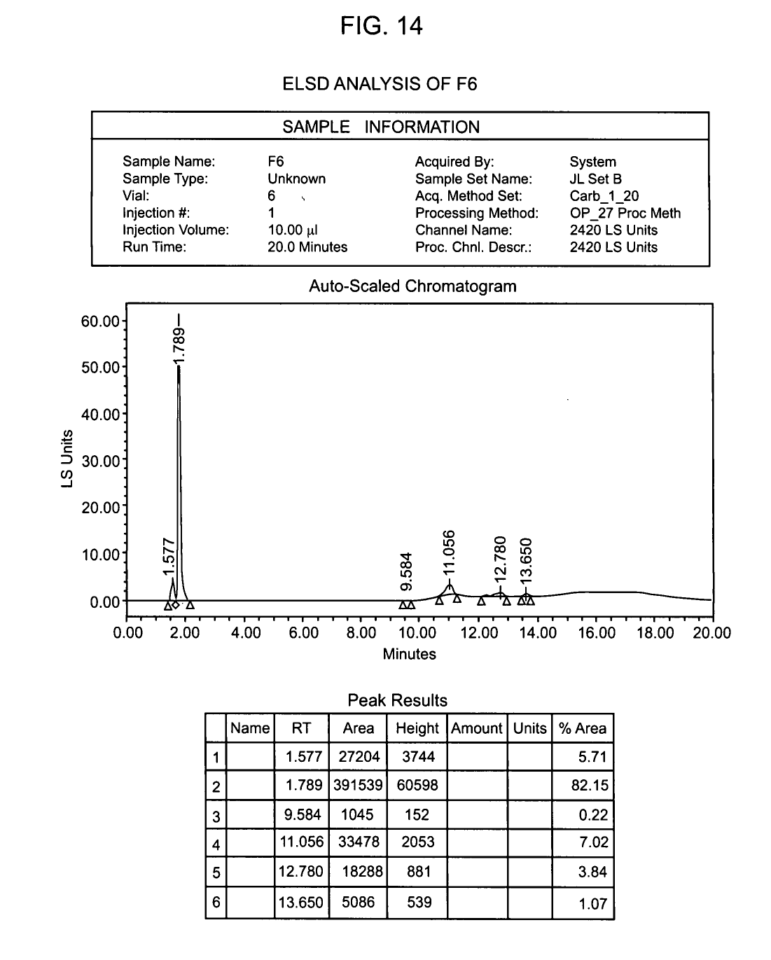

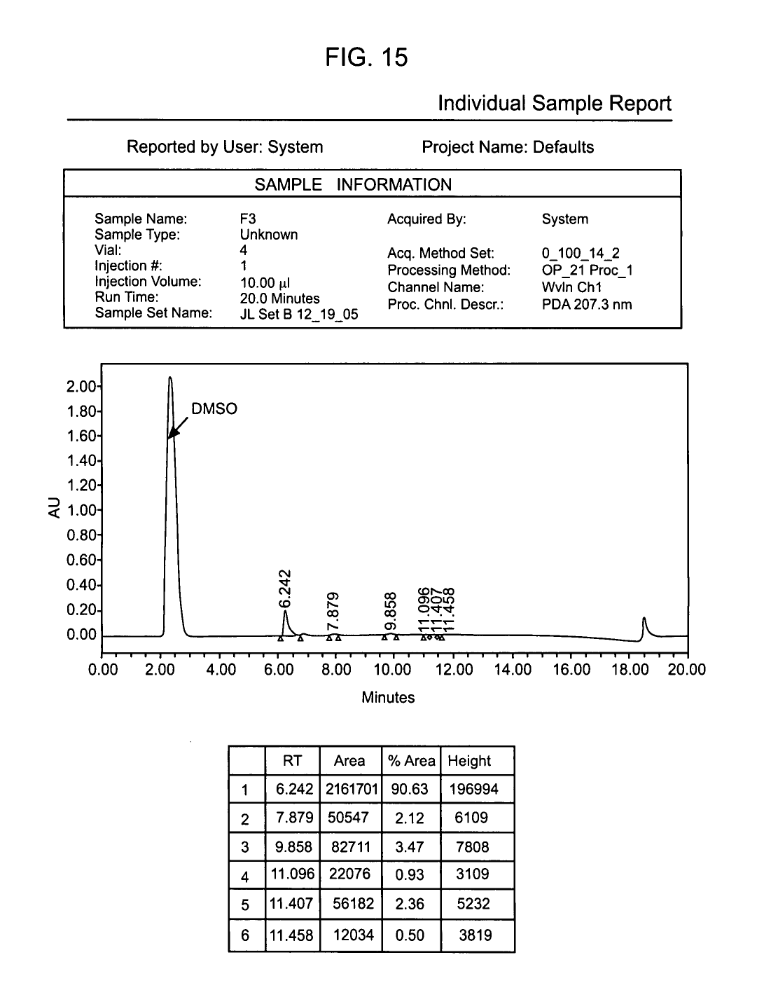

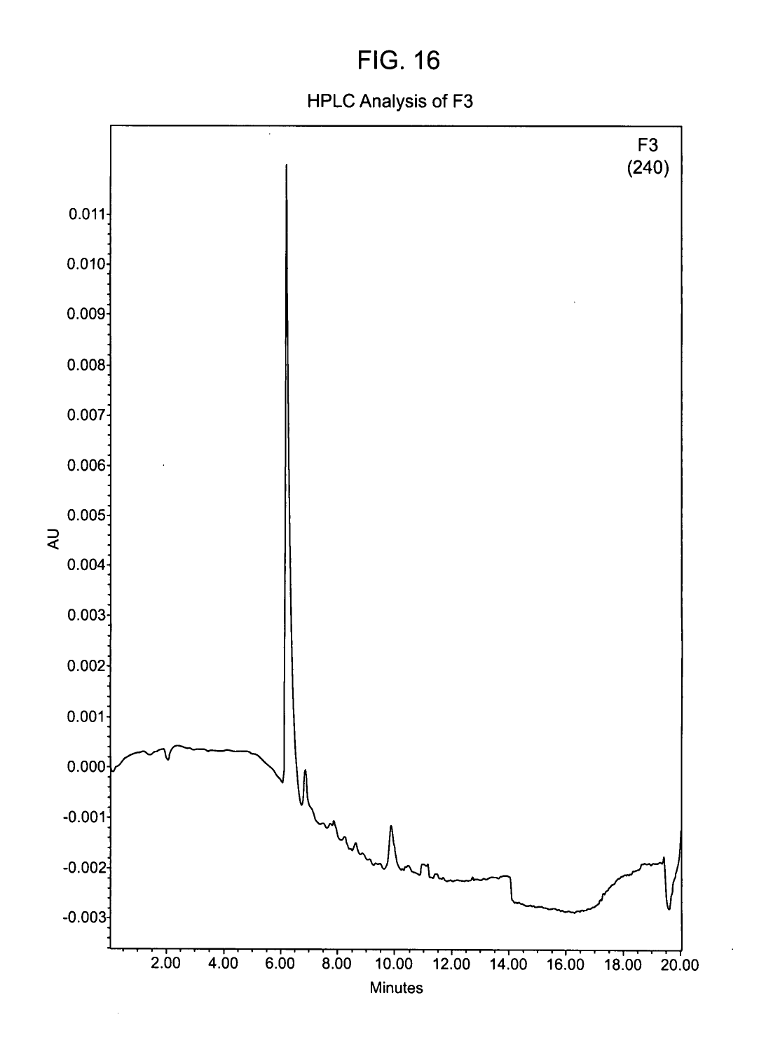

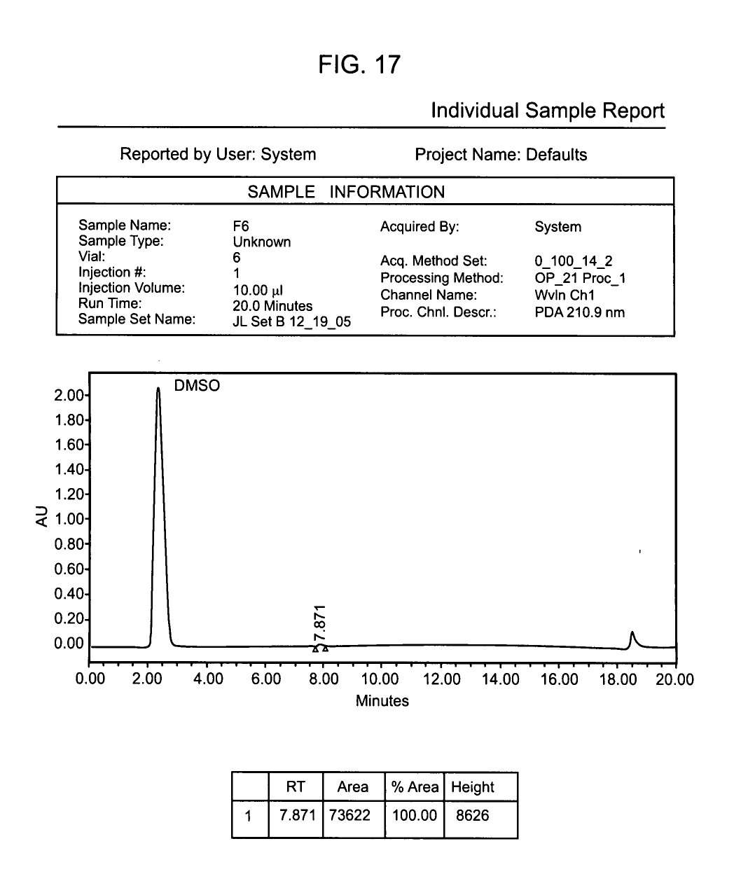

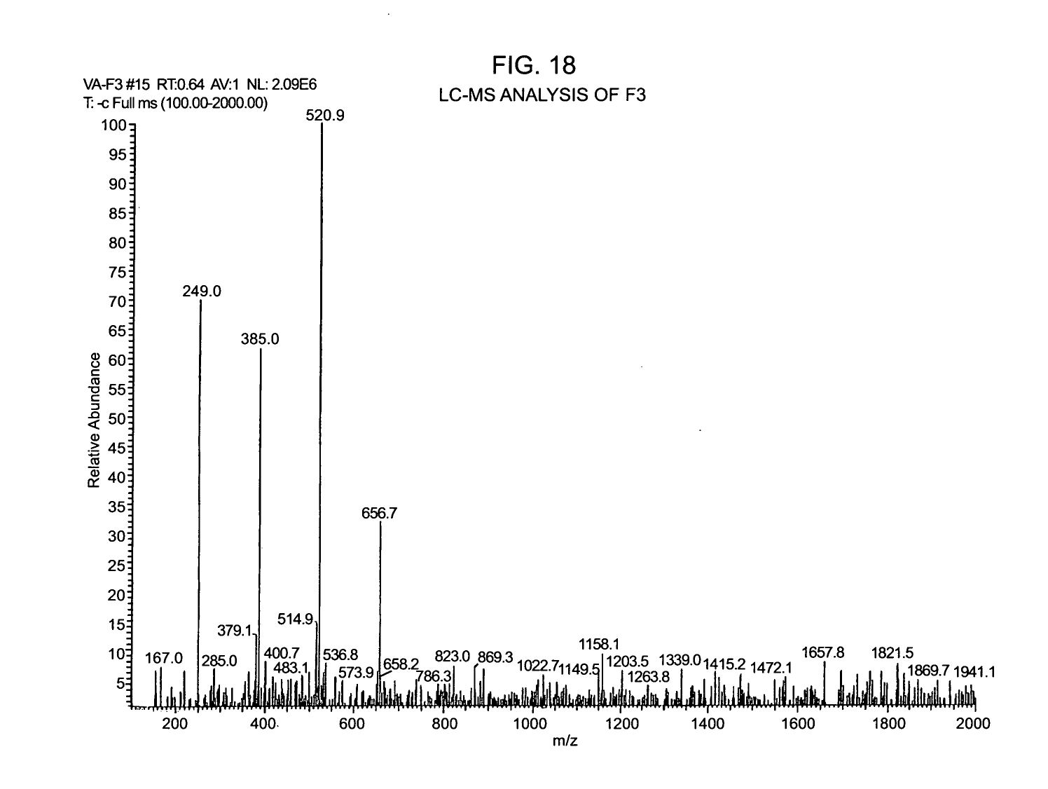

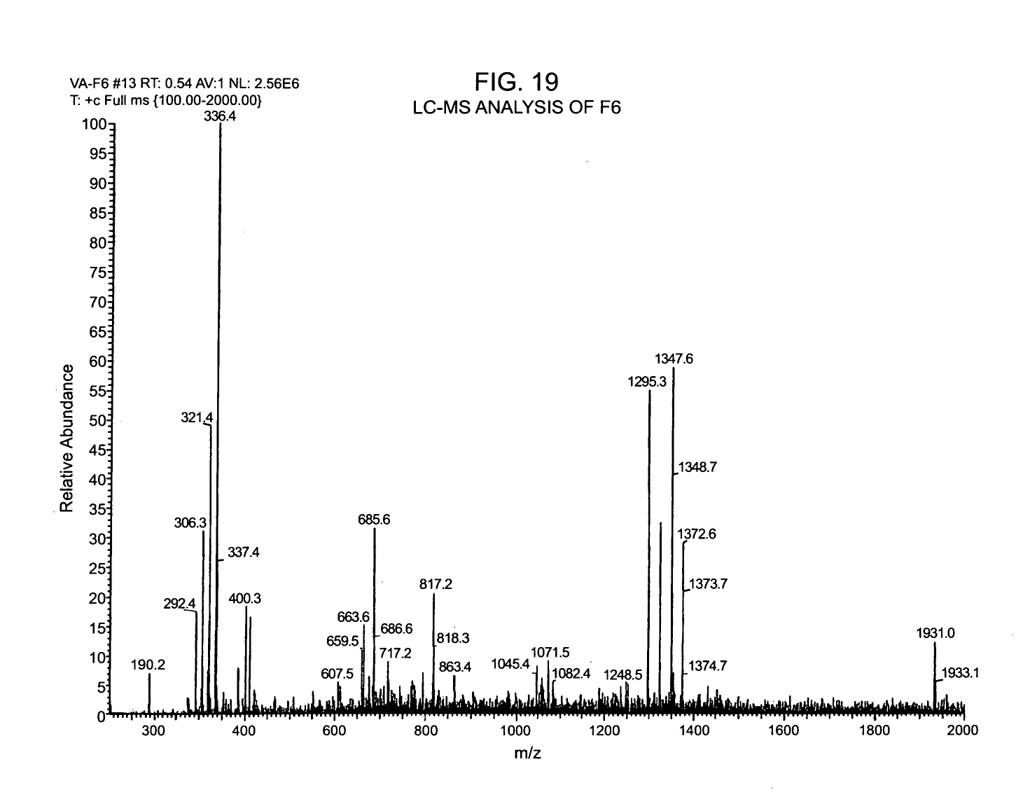

(FIG. 4, Panel B) Fluorescence activated cell sorter (FACS) analysis of intracellular accumulation of BBR: HepG2 cells were incubated with 15 μg/ml of BBR, CND; HDT, or goldenseal (2.2 μ/ml) for 2 h at 37° C. Thereafter, cells were washed with cold PBS and trypsinized. The intracellular fluorescent signal was analyzed by FACS. The MFV of untreated cells is defined as 1 and the MFV in drug treated cells were plotted relative to that value.

(FIG. 4, Panel C) Kinetics of BBR uptake: Cells were incubated with BBR (15 μg/ml) or goldenseal (2.2 μl/ml) at 37° C. At indicated times, medium was removed and cells were collected by trypsinization and were subjected to FACS analysis.

FIG. 5A-5D illustrates that MDRI attenuates BBR intracellular accumulation and BBR activity on LDLR mRNA expression. HepG2 cells were preincubated with 0.6 μM of verapamil (VRMP) for 30 min prior to the addition of BBR or goldenseal. After 2 h drug treatment, the intracellular accumulation of BBR was examined under a fluorescent microscope (FIG. 5A) or was analyzed by FACS (FIG. 5B). In (FIG. 5C), cells were treated with BBR, goldenseal, or CND in the absence or the presence of 0.6 μM VRMP for 8 h. The LDLR mRNA levels were determined by real-time PCR. The fold increase in LDLR mRNA expression was calculated by dividing the activity of each drug in the presence of VRMP to that in the absence of VRMP. The graph shown is summarized results of 3 separate experiments (mean±S.D.). In (FIG. 5D), HepG2 cells were transfected with MDR1 siRNA or a control siRNA for 3 days. The transfected cells were treated with BBR for 6 h. Total RNA was isolated and the mRNA levels. of MDR1, LDLR, and GAPDH were assessed by real-time quantitative RT-PCR.

FIG. 6 illustrates that goldenseal inhibits MDR1 transport activity. Left bar group: HepG2 cells were incubated with 1 μg/ml of DiOC2(3) in the absence or the presence of goldenseal (2.5 μ/ml), VRMP (50 μM), or CND (20 μg/ml) for 2 h at 37° C. The efflux of DiOC2(3) was measured by FACS. Right bar group: HepG2 cells were treated with goldenseal, VRMP, or 1 μM of vinblastine overnight, followed by the addition of DiOC2(3) for 2.5 h. FACS was performed to measure the dye efflux.

FIG. 7A-7F illustrates reduction of cholesterol and lipid accumulation in serum by goldenseal in hypercholesterolemic hamsters:

(FIG. 7A-7D) Serum was taken before, during, and after a 24-day of drug treatment at the indicated doses from hamsters fed a HFHC diet. Results represent mean±S.E.M. of 7-9 animals. In the lower panel, the value in control group at each time point was defined as 100% and the values in treated animals were plotted relative to that value.

(FIG. 7E) After a 24-day treatment, serum lipid levels in treated hamsters were compared to the control animals. Results represent mean±S.E.M. of 7-9 animals. *p<0.01 and **p<0.001, as compared to the values in untreated control group. (FIG. 7F) The final sera from the normal diet group (n=6), the HFHC control group (n=9), and from goldenseal (125 μl/d) group were pooled and the pooled sera were subjected to HPLC analysis of lipoprotein profiles associated with TC and TG.

FIG. 8 illustrates the upregulation of LDLR mRNA expression and activation of ERK signaling pathway in hamsters by goldenseal:

(FIG. 8, Panel A) Hepatic LDLR mRNA expression: 4 h after the last drug treatment, all animals were sacrificed and liver total RNA was isolated. The levels of LDLR mRNA in untreated (n=6), goldenseal (125 μl/d) treated (n=6), and BBR treated (n=6) hamsters fed the HFHC diet were assessed by the quantitative PCR. ***p<0.0001, as compared to control group.

(FIG. 8, Panel B) Western blot of phosphorylated ERK: Cytosolic proteins were prepared from pooled liver samples of the same treatment group (n=9) and 50 μg protein of pooled sample was subjected to SDS-PAGE. The membrane was blotted with anti-phosphorylated ERK antibody, and subsequently blotted with anti-ERK2 antibody.

(FIG. 8, Panel C) Activation of ERK in HepG2 cells: HepG2 cells were treated with 2.5 μ/ml of goldenseal obtained from 3 different suppliers or treated with 20 μg/ml of BBR, HDT, or CND for 2 h. Total cell lysates were prepared and 50 μg protein per sample was analyzed for phosphorylated ERK by western blot analysis.

FIG. 9 illustrates that goldenseal administration reduces hepatic fat storage and eliminates infiltrations of mononuclear leukocytes in hyperlipidemic hamsters. Paraformaldehyde-fixed tissue sections of liver taken from a hamster fed a normal diet (FIG. 9, Panel A), an HFHC diet untreated (FIG. 9, Panel B), an HFHC diet treated with low dose (FIG. 9, Panel C) and high dose (FIG. 9, Panel D) of goldenseal, and BBR (FIG. 9, Panel E) were subjected to H&E and Red Oil O staining. The arrow indicates infiltrating mononuclear leukocytes and the arrowheads indicate portal veins.

FIG. 10 illustrates that goldenseal administration reduces hepatic cholesterol content. The hepatic TC, FC, and TG were measured in hamsters on a normal diet (n=6), a HFHC diet (n=9), and on HFHC diet treated with goldenseal (n=9) or with BBR (n=9). **p<0.001, as compared to HFHC control group. Each column represents the mean±S.D.

FIG. 11 illustrates that goldenseal treatment has no detectable adverse effects. The body weight of hamsters under a HFHC diet in untreated and drug-treated animals at indicated doses were monitored on alternate days (FIG. 11, Panel A). The food intake within 24 h period were measured 2-3 times a week throughout the entire experiment (FIG. 11, Panel B). The data are means±S.D. of 3 cages per group.

FIG. 12 is a graph illustrating canadine (CND) is less toxic to liver cells than berberine (BBR).

FIG. 13 is a graph showing the results of ELSD analysis of F3.

FIG. 14 is a graph showing the results of ELSD analysis of F6.

FIG. 15 is a graph showing the results of HPLC analysis of F3.

FIG. 16 is a graph showing the results of HPLC analysis of F3.

FIG. 17 is a graph showing the results of HPLC analysis of F6.

FIG. 18 is a graph showing the results of LC-MS analysis of F3.

FIG. 19 is a graph showing the results of LC-MS analysis of F6.

### DETAILED DESCRIPTION OF THE INVENTION

The invention features bioactive compounds obtainable from goldenseal and methods of use of such compounds in reducing lipid (at least one of total cholesterol, LDL-cholesterol, free fatty acids, or triglycerides) in a patient having or suspected of having hyperlipidemia,

Before the present invention is described, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Before describing the invention in greater detail, the following definitions are set forth to illustrate and define the meaning and scope of the terms used to describe the invention herein.

As used herein the term “isolated” is meant to describe a compound that is in an environment different from that in which the compound naturally occurs.

The term “substantially pure” as used herein refers to a compound that is removed from its natural environment and is at least 60% free, usually at least 75% free, and more usually at least 90% free from other components with which it is naturally associated. “Substantially pure” compounds are thus compounds of a purity greater than 60%, greater than 75%, such as greater than 80% or 90%, for example, greater than 95%. The present invention is meant to comprehend diastereomers as well as their racemic and resolved, enantiomerically pure forms and pharmaceutically acceptable salts thereof.

The term “in need of treatment” as used herein refers to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregivers expertise, and includes the knowledge that the individual or animal is ill, or will be ill, as the result of a condition that is treatable by the compounds of the invention.

As used herein, the terms “treatment” or “treating” cover any treatment of the disease condition, and include: (1) preventing the disease from occurring in a subject who does not have the disease or who has not yet been diagnosed as having it (e.g., prophylaxis); (2) inhibiting or arresting the development of the disease; or (3) regressing or reversing or alleviating the disease state.

An “effective amount”, “therapeutically effective amount” or “efficacious amount” means the amount of a compound that, when administered to a mammal or other subject for treating a disease, is sufficient to effect such treatment of a disease. The “therapeutically effective amount” will vary depending on the compound, the disease and its severity and the age, weight, etc., of the subject to be treated.

By “lowering” or “reducing” in the context or lowering or reducing serum lipid in a subject (e.g., lowering or reducing total cholesterol, LDL cholesterol, fatty acids, and/or triglycerides) means that the level of serum lipid (e.g., total cholesterol, LDL cholesterol, fatty acids, and/or triglycerides) in the subject following administration of a compound is reduced relative to a pre-treatment serum lipid level (e.g., total cholesterol, LDL cholesterol, fatty acids, and/or triglycerides level). For example, where a compound is administered to reduce LDL cholesterol in a subject, LDL cholesterol levels are reduced in the subject post-treatment as compared to a pre-treatment LDL cholesterol level.

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The term “patient” as used herein refers to any mammal, for example, mice, hamsters, rats, other rodents, rabbits, dogs, cats, swine, cattle, sheep, horses, or primates, including humans. The term may specify male or female or both, or exclude male or female.

The terms “physiologically acceptable,” “pharmaceutically acceptable,” and “pharmaceutical” are interchangeable.

A “pharmaceutically acceptable carrier”, which may be used interchangeably with a “pharmaceutically acceptable diluent” or “pharmaceutically acceptable adjuvant”, refer to substances useful in preparing a pharmaceutical composition that are generally safe, non-toxic and neither biologically nor otherwise undesirable, and include substances acceptable for human use, veterinary use, or both.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to a subject in need thereof via a number of different routes of administration including enteral (e.g., oral, buccal, rectal), parenteral (e.g., intravenous, intraperitoneal, intradermal), pulmonary (e.g., nasal, inhalation, intratracheal), topical, transdermal, and the like.

The term “unit dosage form,” as used herein, refers to physically discrete units suitable as unitary dosages for human and/or non-human animal subjects, each unit containing a predetermined quantity of a compound(s) as disclosed herein calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

### Compounds Present in Goldenseal and Other Than Berberine Have LDLR Regulatory Activity

The present invention stems from the observation that goldenseal root extract has a higher activity in increasing LDLR expression in HepG2 cells than the pure compound berberine (BBR), indicating the presence of other bioactive compounds in goldenseal.

According to the present invention, canadine (CND), has been identified as another major isoquinoline compound of goldenseal and as an inducer of LDLR expression with a greater activity than BBR. It is noteworthy that CND and palmatine (PMT) are structurally closely related to BBR, yet PMT has no regulatory activity on LDLR expression. On the other hand, both BBR and PMT have strong DNA binding affinities, whereas CND, a hydrogenated product of BBR, does not bind to DNA (Qin, Y et al. Bioorganic & Medicinal Chemistry 2006, 14: 25-32). Without being held to theory, the quaternary ammonium and planar structure of BBR and PMT may play important roles in the DNA-binding thereof. The fact that CND lacks both of these important features for DNA binding, but shares the common activity with BBR in stabilizing LDLR mRNA, is indicative that the DNA-binding property is separate from the activity of mRNA stabilization of these isoquinoline compounds.

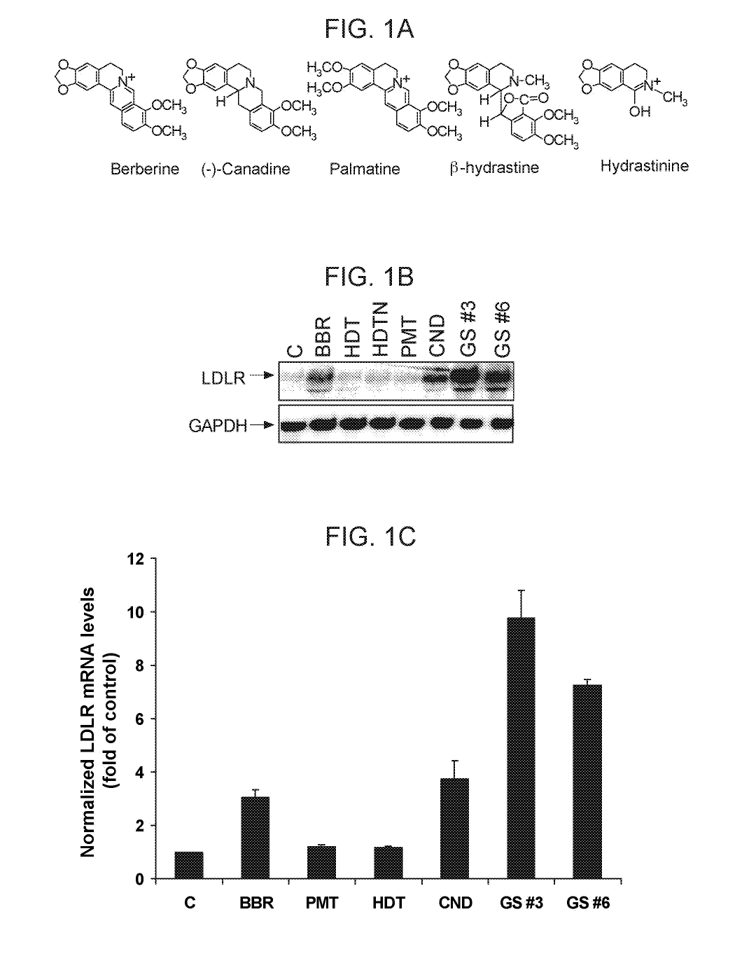

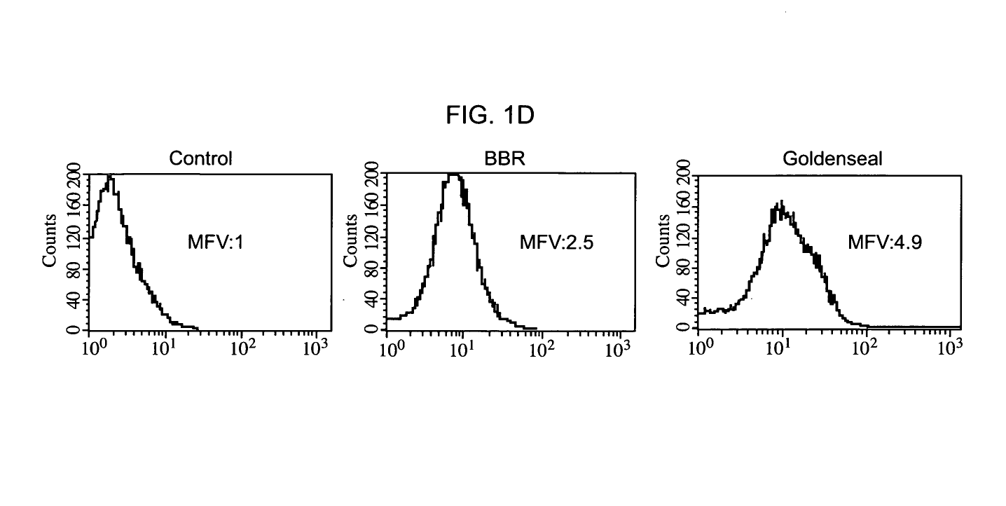

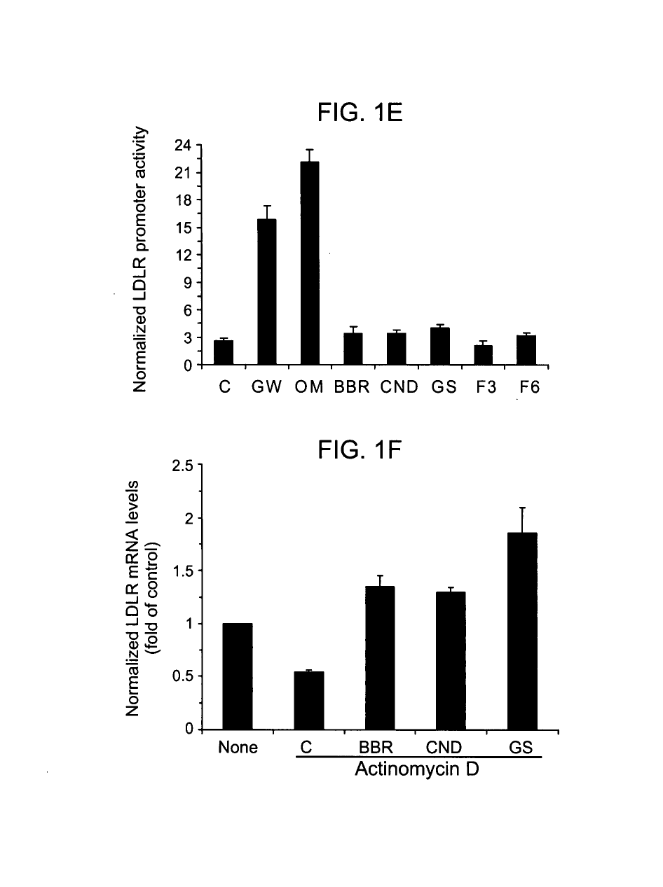

Further, according to the present invention, additional LDLR regulators have been determined to be present in goldenseal extract. Eluate fractions F3 and F6 of silica gel columns loaded with goldenseal have LDLR inducing activities that cannot be attributed either to BBR or to CND. Using HPLC-ELSD detection, fraction F3 was separated into 4 compounds in addition to β-hydrastine (HDT) and fraction F6 was separated into 5 unknown compounds. BBR and CND were absent from F3 or F6. The elevated LDLR expression may be caused by a single compound in F3 or in F6 or may result from a combined action of the mixture. The compound(s) causing LDLR expression in the two fractions are referred to herein as Factor F3 and Factor F6. Since neither fraction F3 nor fraction F6 increased LDLR promoter activity (FIG. 1E), the unknown compound(s) likely act(s) on the stability of LDLR mRNA, although Applicant is in no manner limited by a discussion of this mechanism.

A factor that can contribute to the strong activity of goldenseal in elevating LDLR expression is the resistance of goldenseal to MDR1-mediated (multidrug pump) drug exclusion. By using two different approaches, including MDR1 inhibitors that inhibit the transport activity of MDR1 and siRNA that blocks the expression of MDR1, the present inventors have found that pgp-170 actively excludes BBR from HepG2 cells, which results in a lower efficacy of BBR in LDLR regulation. BBR and palmatine (PMT), which are strong amphipathic cations, have been identified as natural substrates of the MDR NorA pump of microorganisms (Hsieh, P C.et al. Proc. Natl. Acad. Sci. USA 1998, 95: 6602-6606; Stermitz, F R et al. Proc. Natl. Acad. Sci. USA 2000, 97: 1433-1437; Samosorn, S et al. Bioorganic & Medicinal Chemistry 2006, 14: 857-865).

BBR in goldenseal has a longer intracellular retention time, with greater influx and lesser efflux than BBR alone, indicating that MDR inhibitor(s) present in goldenseal act in a synergistic relationship with BBR. As demonstrated in the Examples herein, MDR1-mediated efflux of 3,3′-diethyloxacarbocyanine iodide (DiOC2(3)), a well-characterized MDR1 substrate, is inhibited by goldenseal at the concentration that elicited a response in LDLR expression. Therefore, a further aspect of the present invention is a composition, which is a novel inducer of LDLR expression, that comprises a mixture of BBR and an MDR inhibitor.

Without being held to theory, the observation that CND is not a good substrate of MDR1 supports a molecular explanation for the activity of CND being higher than that of BBR. With its features of MDR1 resistance and reduced (e.g., low or undetectable) DNA binding, CND is a better candidate than BBR alone for clinical use for cholesterol reduction, which may be accompanied by relatively lower toxicity.

The Examples herein further demonstrated strong TC and LDL-c reductions and a 3.2-fold increase in the hepatic LDLR mRNA level in goldenseal treated hamsters fed a high fat high cholesterol (HFHC) diet at half of the equivalent dose of BBR. These in vivo results confirm the higher potency of goldenseal observed in the in vitro studies. In addition to lowering TC and LDL-c, goldenseal and BBR markedly reduced serum FFA and TG.

In addition, goldenseal greatly reduced the lipid accumulation, as well as suppressed the inflammation response, in liver tissue of hamsters given a high fat diet. The high white blood cell count (WBC) caused by the HFHC diet was suppressed to the base line level, which is consistent with the liver histology finding that goldenseal treatment eliminated infiltrations of mononuclear leukocytes.

Various aspects for the practice of the invention are described in more detail below.

### Preparation of the Bioactive Components

The present disclosure provides a method for the preparation of the bioactive compounds of the present invention. Chromatography, specifically preparative high pressure liquid chromatography (HPLC) was used to isolate and separate the bioactive components of a goldenseal root extract from the inactive components. Preparative HPLC is a technique known in the art. Suitable preparative systems include those manufactured by Waters Corporation, Milford, Mass. Using this technique the bioactive components, CND, BBR, and 5 additional active compounds were isolated. The inactive components isolated include β-hydrastine (HDT), hydrastinine (HDTN). As discussed in more detail in the Examples section below, bioactivity of CND, HDT, HTDN, and PMT on LDLR mRNA expression was examined. HPLC analysis demonstrated the presence of BBR, CND, HDT, and HDTN in goldenseal and the absence of PMT. A silica gel column was used to separate goldenseal extract into 26 fractions. Analytical HPLC was used to further separate F3 and F6 into different components, and HPLC-coupled ELSD methods were used to separate F3 and F6 into different peaks.

Methods for producing compounds of interest for use in the methods described herein are described below.

### Canadine (CND)

“Canadine” (or “CND”), also referred to as (d,l)-tetrahydroberberine or Berberis diisoquinoline alkaloid, has the molecular formula C2₀H₂₁NO₄, represented by the structure provided in FIG. 1A (provided below for convenience):

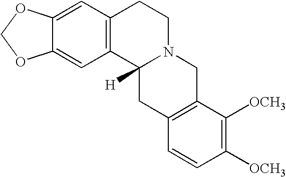

CND useful in the methods described herein can be produced by isolation from a natural source (e.g., isolation from goldenseal) by methods known in the art (e.g., preparative HPLC). CND may also be produced by synthetic methods, e.g., by treating berberine with indium in aqueous ammonium chloride by a method known in the art (see, e.g., Das et al. Synthetic Communications 31:1815-1817 (2001)). Canadine can be also obtained from goldenseal by application of flash chromatography over silica gel with a chloroform methanol 90-50% gradient as an eluting solvent. The fraction containing CND can be further purified by preparative HPLC.

Isolated CND can be provided in a pharmaceutical composition, either as the only active agent or combined with other active agent(s) (e.g., in admixture), where the pharmaceutical composition further contains a pharmaceutically acceptable carrier. Such pharmaceutical compositions generally contain an amount of isolated CND effective to provide for a serum lipid-lowering effect following administration to a subject.

### Eluate Fraction F3

“F3” or “Fraction F3” refers to an isolated fraction of goldenseal which exhibits activity as a serum lipid cholesterol-lowering agent (e.g., as detected by elevated LDLR mRNA levels in a cell (e.g., a liver cell) in the presence of F3 as compared to the absence of F3). F3 is weakly fluorescent. As discussed in the Examples below, F3 was originally isolated as one of twenty-six 15 ml fractions eluted from a standard silica gel column with a chloroform: methanol 10-50% gradient as an eluting solvent. HPLC-coupled evaporative light scattering detection (ELSD) on a normal phase column was used to separate the components of F3 into four compounds in addition to β-hydrastine (HDT). Neither BBR nor CND is one of these compounds. HPLC, ELSD and LC-MS analysis of F3 are provided in FIGS. 13, 15 and 16, and 18. The estimated activity of the active component(s) of F3 is at least about 50 fold to about 100 fold or greater than activity of BBR in modulation of LDLR expression levels.

Isolated F3 or isolated bioactive components of F3 can be provided in a pharmaceutical composition, either as the only active agent or combined with other active agent(s) (e.g., in admixture), where the pharmaceutical composition further contains a pharmaceutically acceptable carrier. Such pharmaceutical compositions generally contain an amount of isolated F3 effective to provide for a serum lipid-lowering effect following administration to a subject.

### Eluate Fraction F6

“F6” or “Fraction F6” refers to an isolated fraction of CND which exhibits activity as a serum lipid cholesterol-lowering agent (e.g., as detected by elevated LDLR mRNA levels in a cell (e.g., a liver cell) in the presence of F6 as compared to the absence of F6). As discussed in more detail in the Examples below, F6 was one of twenty-six 15 ml fractions eluted from a standard silica gel column with chloroform: methanol 10-50% gradient as an eluting solvent. HPLC-coupled evaporative light scattering detection (ELSD) on a normal phase column was used to separate the components of F6 into five compounds, which compounds are distinct from BBR or CND. HPLC, ELSD and LC-MS analyses of F6 are provided in FIGS. 14, 17 and 19. As illustrated in the Examples section below, F6 is a more potent modulator of LDLR expression levels than BBR.

Isolated F6 or isolated bioactive components of F6 can be provided in a pharmaceutical composition, either as the only active agent or combined with other active agent(s) (e.g., in admixture), where the pharmaceutical composition further contains a pharmaceutically acceptable carrier. Such pharmaceutical compositions generally contain an amount of isolated F6 effective to provide for a serum lipid-lowering effect following administration to a subject.

### Berberine (BBR)

BBR may be isolated from goldenseal by preparative HPLC, or may be produced synthetically. BBR is also commercially available from Sigma Chemical Co., St. Louis, Mo. MDR inhibitors are known in the art and are available commercially. Suitable MDR inhibitors include for example, calcium channel blockers, anti-arrhythmics, antihypertensives, antibiotics, antihistamines, immuno-suppressants, steroid hormones, modified steroids, lipophilic cations, diterpenes, detergents, antidepressants, and antipsychotics. See Gottesman, et al. Ann. Rev. Biochem. 1993, 62: 385-427. BBR and an MDR inhibitor are mixed together in a molar ratio of about 90:1, about 85:1, about 80:1, about 75:1, about 70:1, about 65:1, about 60:1, about 55:1, about 50:1, about 45:1, about 40:1, about 35:1, or about 30:, with about 26.8 to 0.6 (about 45:1) being of particular interest, and with a range of from about 90:1 to 15:1, about 85:1 to 20:1 being of interest. Specific molar ratios includes about 26:0.3 (87:1); about 26:0.6 (43:1), and about 26:1 for BBR to MDR inhibitor (e.g., verapamil.

Isolated BBR can be provided in a pharmaceutical composition, either as the only active agent or combined with other active agent(s) (e.g., in admixture), where the pharmaceutical composition further contains a pharmaceutically acceptable carrier. In an embodiment of particular interest, a pharmaceutical composition containing BBR also contains a multi-drug resistance pump inhibitor (MDRI), or the BBR-containing pharmaceutical composition is provided with a separate pharmaceutical composition containing an MDRI (e.g., as separate dosage forms in a kit). In such embodiments, the BBR and MDRI are provided in the pharmaceutical composition (or in each of the separate compositions) in an amount effective to provide for a serum lipid-lowering effect following administration to a subject, where the synergistic effect of combination therapy of BBR and MDRI can be taken into account.

### Multi-Drug Resistance Pump (MDR) Inhibitors

MDR inhibitors (also referred to herein as MDRIs) useful in the methods of the invention include any MDRI which provides for increased retention of a cholesterol-lowering agent, particularly a cholesterol-lowering agent described herein, more particularly BBR. Increased retention of the cholesterol-lowering agent can be assessed as an intracellular level of cholesterol-lowering agent in a cell in the presence of the MDRI as compared to the absence of the MDRI. Exemplary MDRIs which can be used in the methods herein include, verapamil, 5′-methoxyhydnocarpin 5′-MHC), quinidine, quinine, cyclosporine A, VX-710 (in clinic trail), LY335979, R101933, OC144-093, XR9576 and the like. For a review, see, e.g., The Oncologist Vol. 8: 411-424, 2003, which is incorporated herein by reference in its entirety.

MDRIs can be provided as separate pharmaceutical compositions for administration in combination with a lipid lowering agent as described herein, particularly BBR. MDRIs can also be combined formulation with a lipid lowering agent as described herein, particularly with BBR. Such compositions generally contain an amount of MDRI effective to enhance a serum lipid-lowering effect of a compound with which it is administered as part of a combination therapy.

### Pharmaceutically Acceptable Salts, Optical Isomers, and Racemates, and Additional Compounds Contemplated for Use

Compounds for administration in formulations and methods as disclosed herein can employ pharmaceutically acceptable salts, e.g., acid addition or base salts of the compounds. Selection of appropriate salts of the compounds disclosed herein will be readily apparent to the ordinarily skilled artisan, particularly in the pharmaceutical arts. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tal-trate, maleate, fumarate mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. Suitable base salts are formed from bases that form non toxic salts, for example aluminum, calcium, lithium, magnesium, potassium, sodium, zinc and diethanolamine salts.

Particular exemplary pharmaceutically acceptable salts of interest can be prepared by, for example, treating one or more bioactive compounds of the invention that contain a carboxyl moiety with 1-6 equivalents of a base such as sodium hydride, sodium methoxide, sodium ethoxide, sodium hydroxide, potassium tert-butoxide, calcium hydroxide, calcium acetate, calcium chloride, magnesium hydroxide, magnesium chloride, magnesium alkoxide and the like. Solvents such as water, acetone, ether, THF, methanol, ethanol, t-butanol, 2-butanone, dioxane, propanol, butanol, isopropanol, diisopropyl ether, tert-butyl ether or mixtures thereof may be used. Organic bases such as lysine, arginine, methyl benzylamine, ethanolamine, diethanolamine, tromethamine, choline, guanidine and their derivatives may be used. Acid addition salts, wherever applicable, may be prepared by treatment with acids such as tartaric acid, mandelic acid, fumaric acid, malic acid, lactic acid, maleic acid, salicylic acid, citric acid, ascorbic acid, benzene sulfonic acid, p-toluene sulfonic acid, hydroxynaphthoic acid, methane sulfonic acid, acetic acid, benzoic acid, succinic acid, palmitic acid, hydrochloric acid, hydrobromic acid, sulfuric acid, nitric acid and the like in solvents such as water, alcohols, ethers, ethyl acetate, dioxane, THF, acetonitrile, DMF or a lower alkyl ketone such as acetone, or mixtures thereof.

Compounds contemplated for use include, where appropriate, racemates, diastereomers, active isomers (e.g.,. geometric isomers and individual isomers), and enantiomers of the compounds disclosed herein.

The formulations and methods disclosed herein will also be understood to encompass compositions containing a metabolic product of a bioactive compound disclosed herein, where may be generated in vivo after administration of a precursor or parent compound). Such products may result from oxidation, reduction, hydrolysis, amidation, esterification and the like of the administered compound, usually as a result of an enzymatic process(es). Metabolic products of a compound disclosed herein can be generated by administering a parent or precursor compound to a mammal for a period of time sufficient to yield a metabolic product. Metabolic products can be identified by, for example, preparing a radiolabelled parent compound, administering it parenterally in a detectable dose to mammal (usually an animal such as rat, mouse, guinea pig, monkey, or, in some cases, a human), allowing sufficient time for metabolism to occur and isolating conversion products from urine, blood or other biological sample(s).

The methods and formulations of the invention can also include in some embodiments (e.g., in combination therapy with MDR inhibitors), the formulations contemplated for use in the methods herein include one or more of canadaline berberastine; coptisine; dehydrocavidine; dehydroapocavidine; tetradehydroscoulerine; oxyberberine; dihydroberberine; 8-cyanodihydroberberine; tetrahydroberberine N-oxide; N-methyltetrahydroberberinium iodide; berberine betaine; berberrubine; jatrorrhizine; chelerythrine; sanguinarine; I-tetrahydropalmatine; I-stepholidine; discretamine; kikemanine; bharatamine; caseadine; racemate; 2,3-dimethoxyberbine; dehydroapocavidine; dehydrocavidine; dehydrodiscretine; (±)-discretine; dehydrodiscretamine; (±)-discretamine; demethyleneberberine; (s)-(−)-10-demethylxylopinine; dehydropalmatine; karachine; lienkonine; °-methyllienkonine; N-methylsinactine; (S)-(−)-8-Oxotetrahydropalmatine; solidaline; thalifaurine; 2-hydroxy-3-methoxy-10,11-methylenedioxyberberinium; 3-hydroxy-2-methoxy-10,11-methylenedioxyberberine; tetrahydrocorysamine; (+)-Ophiocarpinone; (S)-N-methylcorydalmine; berberine oxime; berberineacetone; berberidic acid; oxyberberine; tetrahydroberberine methiodide; and allocryptopine.

### Pharmaceutical Activity of the Active Components

The active compounds isolated from goldenseal lower the levels of serum lipids, specifically plasma total cholesterol and/or low-density lipoprotein (LDL) cholesterol and/or triglycerides and/or free fatty acids and hence are useful in combating different medical conditions, where such lowering is beneficial. Thus, the compounds may be used to raise the HDL-cholesterol: LDL-cholesterol ratio. The compounds may also be used in the treatment of aberrant cholesterol levels and/or elevated serum lipid levels, such as may be manifested in obesity, hyperlipidemia, hypercholesteremia, hypertension, atherosclerotic disease events, vascular restenosis, diabetes, fatty liver, and many other conditions affected with or by elevated serum lipid levels, specifically elevated total cholesterol, LDL cholesterol, triglycerides, and/or free fatty acids. Typically, the active compounds will comprise an amount that is therapeutically effective, in a single or multiple unit dosage form, over a specified period of therapeutic intervention, to prevent and/or alleviate measurably one or more symptoms of hyperlipidemia or elevated cholesterol.

The active compounds described herein are useful to prevent or reduce the risk of developing conditions that have atherosclerosis resulting from hyperlipidemia as a risk factor, which can lead to diseases and conditions such as atherosclerotic cardiovascular diseases, stroke, coronary heart diseases, cerebrovascular diseases, peripheral vessel diseases and related disorders. The active compounds of this invention are also useful in prevention, halting, controlling, measurably alleviating, or slowing progression or reducing the risk and/or symptoms of the above mentioned disorders along with the resulting secondary diseases such as cardiovascular diseases, like arteriosclerosis, atherosclerosis; diabetic retinopathy, diabetic neuropathy and renal disease including diabetic nephropathy, glomerulonephritis, glomerular sclerosis, nephrotic syndrome, hypertensive nephrosclerosis and end stage renal diseases, like microalbuminuria and albuminuria, which may be result of hyperglycemia or hyperinsulinemia. In some embodiments, the subject is one who is not an arrhythmia patient, or is other than a subject diagnosed as having or suspected of having an arrhythmia and/or the bioactive compound(s) disclosed herein is not administered as an anti-arrhythmia agent. In some embodiments, the bioactive compound(s) is not administered as an antibiotic. As used herein, the term “cardiovascular disease” is intended to include a range of symptoms, conditions, and/or diseases including atherosclerosis, coronary artery disease, angina pectoris, carotid artery disease, strokes, cerebral arteriosclerosis, myocardial infarction, high blood pressure, cerebral infarction, restenosis following balloon angioplasty, intermittent claudication, dyslipidemia post-prandial lipidemia and xanthoma, and all conventionally targeted symptoms arising from or associated with the foregoing diseases and conditions. Exemplary symptoms of cardiovascular disease can include shortness of breath, chest pain, leg pain, tiredness, confusion vision changes, blood in urine, nosebleeds, irregular heartbeat, loss of balance or coordination, weakness, or vertigo.

The compositions also find use in controlling hyperlipidemia, e.g., so as to provide for a decrease in serum lipid levels in subject having hyperlipidemia, and can include lowering to and/or maintaining serum lipid levels within an acceptable range (e.g., within a range considered normal). The compositions also find use in modulating LDLR expression in a patient and/or provide for increasing LDLR mRNA stability in a cell of patient. The compositions also find use in modulating ERK activation in a patient. Regardless of the mechanism involved, the therapeutic goals of modulation of LDLR expression, increasing LDLR mRNA stability, and/or modulating ERK activity are in concordance with the therapeutic goals described herein in general.

Subjects of interest for the methods of the invention are primarily human patients. Such patients typically have undesirably high levels of serum lipids, which may be defined by total cholesterol (TC), LDL cholesterol, fatty acids, or triglycerides. Normal serum lipid levels, including normal plasma TC levels, LDL cholesterol levels, fatty acids levels, and triglyceride levels, generally refer to those levels recognized as desirable in the relevant clinical fields, and can vary according to age, gender, pre-existing condition, family or genetic history of disease, ethnic origin, and the like, and are subject to change as the understanding in the field improves with respect to such levels as risk factor for or indications of disease. For example, prospective studies have shown that the incidence of coronary artery disease (CAD) rises continuously with plasma TC and that values previously considered normal in the USA are higher than those found among populations with a low incidence of atherosclerosis. In addition, evidence shows that lowering even average American levels of TC (and LDL) in patients with CAD slows or reverses the progression of CAD.

The optimal plasma TC for a middle-aged adult free of CAD is probably less than about 200 mg/dL. Some studies have defined TC levels <200 mg/dL as desirable, levels between 200 and 240 mg/dL as borderline high, and levels >240 mg/dL as high. Other studies have shown a benefit to patients with CAD in reducing TC levels to considerably lower levels, such as less than 100 mg/dL for patients at risk of CAD, usually less than 70 mg/dL for patients with active disease, although high HDL level (>60 mg/dL) is considered a negative risk factor and reduces the number of risk factors.

It is often recommended that treatment decisions be based on the calculated level of LDL. For patients with an elevated LDL L≦160 mg/dL) who have fewer than two risk factors in addition to elevated LDL and who do not have clinical evidence of atherosclerotic disease, the goal of treatment is an LDL level <160 mg/dL. For those who have at least two other risk factors, the goal of treatment is an LDL level <130 mg/dL. When LDL levels remain >160 mg/dL despite dietary measures and the patient has two or more risk factors (in addition to high LDL), or when LDL levels remain >190 mg/dL even without added risk factors, the addition of drug treatment should be considered. For those with CAD, peripheral vascular disease, or cerebrovascular disease, the goal of treatment is an LDL <100 mg/dL.

A useful clinical appraisal of lipids can usually be made by determining plasma TC, HDL-cholesterol, fatty acids, and/or triglyceride levels after the patient has fasted for at least 12 h or more. Plasma TC may be determined by calorimetric, gas-liquid chromatographic, enzymatic, or other automated “direct” methods. Enzymatic methods are usually accurate and are standard in virtually all clinical laboratories. Plasma triglyceride is usually measured as glycerol by either calorimetric, enzymatic, or fluorometric methods after alkaline or enzymatic hydrolysis to glycerol and formaldehyde. HDL levels can be measured enzymatically after precipitation of VLDL, IDL, and LDL from plasma. These and other methods of assessing serum lipids can be used both to identify subject suitable for treatment according to the methods described herein, as well as to monitor response to therapy (e.g. to provide for adjusting of dose, frequency of dose, and the like).

### Pharmaceutical Formulations

The present invention provides pharmaceutical compositions, containing at least one of substantially pure canadine, at least one other hypolipidemic and/or hypocholesteremic compound, other than berberine, isolated from goldenseal, or substantially pure berberine in combination with a multidrug pump inhibitor, their derivatives, their analogs, their tautomeric forms, their stereoisomers, their pharmaceutically acceptable salts, and their pharmaceutically acceptable solvates thereof as an active ingredient, together with pharmaceutically acceptable carriers, diluents, and the like.

Pharmaceutical compositions containing a hypolipidemic and/or hypocholesteremic compound of the present invention may be prepared by conventional techniques, e.g., as described in Remington: the Science and Practice of Pharmacy, 19th Ed., 1995. The compositions may be in the conventional forms, such as capsules, tablets, powders, solutions, suspensions, syrups, aerosols, or topical applications. They may contain suitable solid or liquid carriers or may be in suitable sterile media to form injectable solutions or suspensions. The compositions may contain 0.5 to 20%, preferably 0.5 to 10% by weight of the active compound, the remaining ingredients being pharmaceutically acceptable carriers, excipients, diluents, solvents, and the like.

Typical compositions contain a hypolipidemic and/or hypocholesteremic compound according to the present invention or a pharmaceutically acceptable salt thereof, associated with one or more pharmaceutically acceptable excipients, which may be a carrier or a diluent or may be diluted by a carrier, or may be enclosed within a carrier, which can be in the form of a capsule, sachet, paper or other container. When the carrier serves as a diluent, it may be a solid, semi-solid, or liquid material, which acts as a vehicle, excipient, or medium for the active compound. The active compound can be absorbed on a granular solid container for example in a sachet. Suitable carriers include water, salt solutions, alcohols, polyethylene glycols, polyhydroxyethoxylated castor oil, peanut oil, olive oil, gelatin, lactose, terra alba, sucrose, cyclodextrin, amylose, magnesium sterate, talc, gelatin, agar, pectin, acacia, stearic acid or lower alkyl ethers of cellulose, silicic acid, fatty acids, fatty acid amines, fatty acids monoglycerides and diglycerides, pentaerythritol fatty acids esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preservatives, sweetening agents, or flavoring agents. The formulations of the invention may be formulated so as to provide quick, sustained, or delayed release of the active ingredient after administration to the patient by employing procedures well known in the art. The pharmaceutical compositions can be sterilized and mixed, if desired, with auxiliary agents, emulsifiers, buffers and/or coloring substances and the like, which do not react deleteriously with the active compounds.

Compositions of the invention can also include, as appropriate for the bioactive compound, dosage form and route of administration, binders, fillers, lubricants, emulsifiers, suspending agents, sweeteners, flavorings, preservatives, buffers, wetting agents, disintegrants, effervescent agents and other conventional excipients and additives.

The compositions disclosed herein can be administered in a sustained release form by use of a slow release carrier, such as a hydrophilic, slow release polymer. Exemplary sustained release agents in this context include, but are not limited to, hydroxypropyl methyl cellulose, having a viscosity in the range of about 100 cps to about 100,000 cps or other biocompatible matrices such as cholesterol.

The compositions disclosed herein can also be, and often will be, formulated and administered in an oral dosage form, optionally in combination with a carrier or other additive(s). Suitable carriers can be selected, with exemplary carriers including, but not limited to, microcrystalline cellulose, lactose, sucrose, fructose, glucose, dextrose, or other sugars, di-basic calcium phosphate, calcium sulfate, cellulose, methylcellulose and derivatives thereof, kaolin, mannitol, lactitol, maltitol, xylitol, sorbitol, or other sugar alcohols, dry starch, dextrin, maltodextrin or other polysaccharides, inositol, or mixtures thereof. As discussed infra, exemplary unit oral dosage forms include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants.

Inhalation and nasal delivery dosage forms are also contemplated. Devices suitable for delivering a dry or wet aerosolized formulation include, but are not necessarily limited to, metered dose inhalers, nebulizers, dry powder generators, sprayers, and the like. Further suitable formulations include nasal formulations, such as a nasal spray, may include aqueous or oily solutions of one or more bioactive compounds disclosed herein and additional active or inactive compounds.

Topical compositions for delivery to skin or mucosa are also contemplated. In such compositions, one or more bioactive compounds are formulated with a carrier suitable for dermatological or mucosal delivery. Exemplary topical dosage forms include aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. Delivery of the bioactive compound may be enhanced by use of a dermal or mucosal penetration enhancer.

Parenteral formulations (e.g., for administration intravenously, intramuscularly, subcutaneously, or intraperitoneally, and the like) include aqueous and non-aqueous sterile injectable solutions. Parenteral formulations, like all other formulations disclosed herein, can contain additional active or inactive compounds. For example, parenteral formulations may include buffers, antibiotics, and/or solutes which render the formulation isotonic with the blood of the subject; and aqueous and non-aqueous sterile suspensions which may include suspending agents and/or thickening agents. The formulations may be presented in unit-dose or multi-dose containers.

Pharmaceutically acceptable formulations and components thereof will typically be sterile or readily sterilizable. Parenteral preparations and selected other preparations contain buffering agents and preservatives, and injectable fluids that are pharmaceutically and physiologically acceptable such as water, physiological saline, balanced salt solutions, aqueous dextrose, glycerol or the like. Injection solutions, emulsions and suspensions may be prepared from sterile powders, granules and tablets of the kind previously described. Unit dosage forms of particular interest are those containing a daily dose or unit, daily sub-dose, or other appropriate fraction of a therapeutic dose of the bioactive compound(s).

The compound(s) in the formulation can be provided in a variety of different physical forms, which will be selected according to, for example, the route of administration and the like. For example, the bioactive compound(s) can be provided in the form of microcapsules(including gelatin-microcapsules and poly(methyl methacylate) microcapsules), microparticles, or microspheres, and may be provided as colloidal drug delivery systems (for example, liposomes, albumin microspheres, microemulsions, nanoparticles and nanocapsules); or within macroemulsions.

### Routes of Administration

The methods involving administration of a cholesterol-lowering agent as described herein can be accomplished in a variety of ways. The route of administration may be any route that transports the active drug effectively to provide a desired effect. The methods disclosed herein can be accomplished by any suitable route of administration including enteral, parenteral, pulmonary, topical (e.g., to skin), mucosal, transdermal, and the like. Further exemplary routes include oral, buccal, rectal, intravenous, subcutaneous, intramuscular, intranasal, intraperitoneal, intradermal, nasal, inhalation, in tracheal, intraurethral, and intraocular. Dosage forms for delivery can be selected as appropriate taking into consideration, e.g., the compound and formulation to be delivered, the route of administration, and the like. The dosage form can be provided as a depot, aerosol, injectable, slow release (e.g., sustained release or controlled release), iontophoretic, sonophoretic, or other dosage form.

If a solid carrier is used for oral administration, the preparation may be tabletted, placed in a hard gelatin capsule in powder or pellet form, or shaped in the form of a troche or lozenge. If a liquid carrier is used, the preparation may be in the form of a syrup, emulsion, soft gelatin capsule, or sterile injectable liquid such as an aqueous or non-aqueous liquid suspension or solution.

For nasal administration, the preparation may contain a compound of the invention dissolved or suspended in a liquid carrier, for example an aqueous carrier, for aerosol application. The carrier may contain additives such as one or more solubilizing agents, e.g., propylene glycol, surfactants, absorption enhancers such as lecithin (phosphatidylcholine) or cyclodextrin, or preservatives such as parabens.

For parental application, injectable solutions or suspensions, for example, are suitable, including aqueous solutions with the active compound dissolved in polyhydroxylated castor oil.

For oral administration, tablet, dragées, or capsules having talc and/or a carbohydrate carrier or binder or the like are suitable. Suitable carriers for tablets, dragées, or capsules include lactose, corn starch, and/or potato starch. A syrup or elixir can be used in cases where a sweetened vehicle is to be employed. A typical tablet which may be prepared by conventional tabletting techniques may contain: Active compound (as free compound or salt thereof) (e.g., 5.0 mg); Colloidal silicon dioxide (1.5 mg); Cellulose, microcrystalline (e.g., 70.0 mg); Modified cellulose gum (e.g., 7.5 mg); Magnesium stearate (ad.) and coating composed of Hydroxypropylmethylcellulose (e.g., approx. 9.0 mg); Acylated monoglyceride (used as a plasticizer for film coating) (e.g., MYWACETT 9-40 T™) (e.g., approx. 0.9 mg).

The compounds and/or the compositions of the present invention are useful for the treatment (including prevention) of disease caused by metabolic disorders such as hyperlipidemia, insulin resistance, Leptin resistance, hyperglycemia, obesity, or inflammation. Further, the compounds and/or compositions are useful for the treatment of hyperlipidemia, hypercholesteremia, familial hypercholesteremia, hypertriglyceridemia, type 2 diabetes, dyslipidemia, obesity, insulin resistance, coronary heart disease, atherosclerosis, xanthoma, stroke, peripheral vascular diseases and related disorders, and diabetic complications.

The compounds and/or compositions of the invention may be administered to a mammal, including a human in need of such treatment, including prevention, elimination, alleviation, or amelioration, of the diseases mentioned above.

The compositions of the invention are provided in a dose and for a period of time sufficient to reduce a serum lipid as desired (e.g., total serum cholesterol, LDL cholesterol, fatty acids, triglycerides). Such reduction may be at least about 2.5% of the original starting level, at least about 5%, at least about 7.5%, at least about 10%, at least about 15%, at least about 20%, or more. In some instances, the reduction of total serum cholesterol is at least about 30%, at least about 40%, at least about 50%, or more. The reduction in cholesterol may also be achieved by combining the compositions of the invention with a second cholesterol lowering agent, e.g. statin, fibrate, and the like.

The compounds and/or compositions of the present invention can be effective over a wide dosage range. However, the exact dosage, mode of administration, and form of composition depend upon the patient to be treated and are determined by the physician or veterinarian responsible for treating the patient. Generally, dosages from about 0.025 mg to about 200 mg per day, including from about 0.1 mg to about 100 mg per day, may be used. The compounds and/or compositions may be administered in unit dosage form comprising about 0.01 mg to 100 mg of the active ingredient together with a pharmaceutically acceptable carrier. Generally, suitable dosage forms for nasal, oral, transdermal, or pulmonary administration comprise from about 0.001 mg to about 100 mg, including from 0.01 mg to about 50 mg, of the active ingredient mixed with a pharmaceutically acceptable carrier or diluent.

The bioactive lipid-lowering compound(s) disclosed herein can be administered multiple doses. For example, the compound(s) can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Frequently the course of treatment will continue for extended periods of time, including weeks, months and even years. During this time, the pharmaceutical compositions may be administered weekly, daily, twice daily, or in divided doses as appropriate for the specific formulation. In some instances the course of treatment will be discontinued after one, two or more weeks, based on patient improvement, side effects, and the like.

The methods of the invention also include treatment (including prevention) of the diseases mentioned herein, or, alternatively production of an anti-dyslipidemic, LDL-modulatory, or LDLR-modulatory response in a cell, tissue, organ, or patient. Further, the invention contemplates the use of one or more compounds disclosed herein, or a pharmaceutically acceptable salt thereof, for the preparation of a medicament for the treatment (including prevention) of the diseases and/or responses mentioned above.

Combination Therapies

The bioactive compounds disclosed herein are useful in the methods of the invention alone or in combination with other agent(s) which can provide for additive or synergistic effects in therapeutic response, or provide some other therapeutic benefit.

Exemplary agents for administration in combination with one or more; two or more; or three or more of the bioactive serum lipid-lowering compounds as described herein include one or more of a hypoglycemic agents; antihyperglycemic agents; hypolipidemic agents; agents useful for treating cardiovascular disease; hypolipoproteinemic agents; antioxidants; cholesterol uptake inhibitors; cholesterol biosynthesis inhibitors (such as HMG CoA reductase inhibitor, including statins); HMG-CoA synthase inhibitors; glitazones; sulfonyl ureas; insulin; α-glycosidase inhibitors; cholestipol; cholestyramine; probucol; biguanides; angiotensin II inhibitors; aspirin; insulin secretagogue; β-sitosterol inhibitor; sulfonylureas; insulin; fibric acid derivatives; squalene epoxidase inhibitors or squalene synthetase inhibitors (also known as squalene synthase inhibitors); acyl-coenzyme A cholesterol acyltransferase (ACAT) inhibitors (e.g., melinamide); nicotinic acid and the salts thereof; niacinamide; cholesterol absorption inhibitors (e.g., 6-sitosterol or ezetimibe); bile acid sequestrant anion exchange resins (e.g., cholestyramine, colestipol, colesevelam or diallqlaminoalkyl derivatives of a cross-linked dextran); LDL receptor inducers; fibrates (e.g., clofibrate, bezafibrate, fenofibrate and gemfibrozil); vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, provided above) anti-oxidant vitamins (e.g., vitamin C, vitamin E and betacarotene); β-blockers; angiotensin-converting enzyme inhibitors, renin inhibitors; platelet aggregation inhibitors (e.g., fibrinogen receptor antagonists (glycoprotein IIb/IIIa fibrinogen receptor antagonists); hormones (e.g., estrogen); insulin; omega-3 oils; benfluorex; ethyl icosapentate; and amlodipine. Adjunctive therapies may also include increases in exercise, surgery, and changes in diet (e.g., to a low cholesterol diet). Some herbal remedies may also be employed effectively in combinatorial formulations and coordinate therapies for treating hyperlipidemia, for example curcumin, gugulipid, garlic, vitamin E, soy, soluble fiber, fish oil, green tea, camitine, chromium, coenzyme Q 10, anti-oxidant vitamins, grape seed extract, pantothine, red yeast rice, and royal jelly.

In some embodiments, a bioactive compound described herein (e.g., CND, F3, F6) is administered in combination with at least a second serum lipid-lowering agent (e.g., a non-canadine lipid lowering agent). The second serum lipid-lowering agent can be a bioactive compound described herein, or a serum lipid-lowering agent known in the art. A variety of serum lipid- lowering agents are known in the art, including statins, fibrates, nicotinic acid, sequestering agents, etc. Of particular interest are use of serum lipid-lowering agents that act through a mechanism different from that of the compounds described herein. For example, where the compound is CND, then administration of an agent that acts by affecting LDLR transcription levels (e.g., a statin) is of particular interest.

The agents can be provided as a combination therapy by incorporation into a variety of formulations for therapeutic administration, or can be provided as separate dosage forms in a kit. The agents can be delivered simultaneously or at different times (usually within a relatively short period of time between administrations), and can be administered by the same or by different routes. In some embodiments, a co-formulation is used, where the two components are combined in a single suspension (e.g., by admixture). Alternatively, the agents are separately formulated. The combined effect may be additive, or may provide for a synergistic effect.

Part of the total dose may be administered by different routes. Such administration may use any route that results in systemic absorption, by any one of several known routes, including but not limited to oral administration, inhalation, i.e. pulmonary aerosol administration; intranasal; sublingually; and by injection, e.g. subcutaneously, intramuscularly, etc.

Where the second serum lipid-lowering agent is a statin, the statin can be selected from any of a variety of statin-based therapies. In general “statins” refers to a known class of HMG-CoA reductase inhibitors. These agents are described in detail, for example, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional compounds are disclosed in U.S. Pat. Nos. 5,208,258, 5,130,306, 5,116,870, 5,049,696, RE 36,481, and RE 36,520. Rosuvastatin has been commercialized. Further statins include pitavastatin, and atorvastatin. The lipophilicity of certain statins make them particularly suitable for subcutaneous delivery.

Other agents which may be of interest for use in combination therapy with a bioactive compound described herein include bile acid sequestrants. These drugs bind with cholesterol-containing bile acids in the intestines and are then eliminated in the stool. The usual effect of bile acid sequestrants is to lower LDL-cholesterol by about 10 to 20 percent. Small doses of sequestrants can produce useful reductions in LDL-cholesterol. Cholestyramine, colestipol, and colesevelam are the three main bile acid sequestrants currently available. These three drugs are available as powders or tablets.

Nicotinic acid or niacin, the water-soluble B vitamin, improves all lipoproteins when given in doses well above the vitamin requirement, and can be suitable for use in combination therapy with the bioactive compounds of the invention. Nicotinic acid lowers total cholesterol, LDL-cholesterol, and triglyceride levels, while raising HDL-cholesterol levels. There are three types of nicotinic acid: immediate release, timed release, and extended release. Patients on nicotinic acid are usually started on low daily doses and gradually increased to an average daily dose of 1.5 to 3 grams per day for the immediate release form and 1.5 to 2 grams per day for the other forms. Nicotinic acid reduces LDL-cholesterol levels by 10 to 20 percent, reduces triglycerides by 20 to 50 percent, and raises HDL-cholesterol by 15 to 35 percent.

Fibric acid (or fibrates) work by reducing triglyceride production and removing triglycerides from circulation, and can be of interest for use in combination therapy with the bioactive compounds of the invention. These triglyceride-lowering drugs also increase the levels of HDL “good” cholesterol. Fibrates include gemfibrozil (Lopid) and fenofibrate (Tricor).

### Kits

Kits with unit doses of the subject compounds, e.g., in oral or injectable doses, are provided. In such kits, in addition to the containers containing the unit doses will be an informational and/or instructional package insert describing the use and attendant benefits of the drugs in treating a pathological condition of interest. Preferred compounds and unit doses are those described herein above.

### EXAMPLES

The following examples are considered illustrative, and thus are not limiting of the remainder of the disclosure in any way whatsoever.

### Methods and Materials

The following methods and materials were used in the Examples below.

Analysis and quantitation of alkaloid components in goldenseal. BBR, (−)-CND, p-HDT, PMT, and HDTN were purchased from Sigma Chemical Co. and stock solutions of 10 mg/ml in DMSO were used as standard in HPLC, ELSD, and LC-MS. Goldenseal root extract in 60% grain alcohol contains 200 mg/ml herb weight equivalence was diluted in methanol and subjected to HPLC, ELSD, and LC-MS to determine the alkaloid contents. Direction was given to Combinix Inc. in Mountain View, Calif. to perform the chemical analysis.

Quantitation of LDLR mRNA expression by northern blot analysis and real-time PCR. Isolation of total RNA and analysis of LDLR and GAPDH mRNA by northern blot were performed as previously described (Liu, J et al. J. Lipid Res. 1997, 38: 2035-2048). Differences in hybridization signals of northern blots were quantitated by a PhosphoImager. For quantitative real-time PCR assays, the reverse transcription was conducted with random primers using M-MLV (Promega) at 37° C. for 1 h in a volume of 25 u.1 containing 1 μg of total RNA. Real-time PCR was performed on the cDNA using ABI Prism 7900-HT Sequence Detection System and Universal MasterMix. The human and hamster LDLR and GAPDH Pre-Developed TaqMan Assay Reagents (Applied Biosystems) were used to assess the mRNA expressions in HepG2 and in hamster livers. The MDR1 mRNA expression in HepG2 cells was assayed similarly using the Pre-Developed probes from Applied Biosystems.

LDL uptake assay. HepG2 cells in 6-well culture plates were treated with compounds for 18 h. The fluorescent Dil-LDL (Biomedical Technologies, Stoughton, Massachusetts) at a concentration of 6 (μg/ml was added to the cells at the end of treatment for 4 h and cells were trypsinized. The mean red fluorescence of 2×10⁴ cells was measured using FACScan (filter 610/20 DF, BD LSRII, Becton Dickinson).

Transient transfection and dual luciferase reporter assays. HepG2 cells were transfected with plasmid DNA (100 ng/well) by using FuGENE 6 transfection reagent. The DNA ratio of pLDLR234Luc (Li, C et al. J. Biol. Chem. 1999, 274: 6747-6753) to renilla luciferase reporter pRL-SV40 was 90:10. Twenty h after transfection, medium was changed to 0.5% FBS and drugs were added for 8 h prior to cell lysis. The luciferase activity in cell lysate was measured using Dual Luciferase Assay System obtained from Promega. Triplicate wells were assayed for each transfection condition.

Semi-purification of goldenseal alkaloid components. 1 ml of goldenseal liquid extract was subjected to flash chromatography over silica gel column with chloroform: methanol 10-50% gradient as an eluting solvent. Twenty-six 15 ml fractions were collected. 200 μl of each fraction was directly used to measure the fluorescent intensity with a fluorescent microplate reader (Spectra MaxGemini, Molecular Devices, Sunnyvale, Calif.) at 350-nm excitation and 545-nm emission. Rest of the fraction was evaporated under N2 and residues in each fraction were dissolved in 250 μl of DMSO. 10 μl from each fraction was diluted with 90 μl ethanol and was applied to HPLC, ELSD, and LC-MS respectively.

BBR uptake assay. HepG2 cells were seeded in 6-well culture plates at a density of 0.8×10⁶ cells/well in medium containing 10% FBS. Next day, cells were incubated with medium without serum. BBR at a concentration of 15 μg/ml or goldenseal with equivalent amount of BBR were added to the cells for the indicated times. At the end of treatment, cells were washed with cold PBS and trypsinized. The cell suspensions in PBS were placed on ice to minimize efflux activity. The mean green fluorescence of 2×10⁴ cells was measured using FACScan (filter 525/50HQ, BD LSRII, Becton Dickinson).

MDR direct dye efflux assay. The MDR Direct Dye Efflux Assay kit (Cat. No. ECM910, Chemicon International Inc., Temecula, Calif.) was used to measure MDR1 activity. HepG2 cells seeded in 6-well culture plates were incubated in efflux buffer (RPMI+2% BSA) and 1 μg/ml of DiOC2(3) in the absence of presence of tested compounds at 37° C. for 2 h. Cells were washed with cold PBS and trypsinized. The cell suspensions in PBS were placed on ice to minimize efflux activity. The mean green fluorescence of 2×10⁴ cells was measured using FACScan (filter 530/30DF, BD LSRII, Becton Dickinson). The DiOC2(3) efflux assay was also performed in HepG2 cells that were pretreated with goldenseal, vinblastine, or VRPM to inhibit the MDR1 transport activity. The weak green fluorescence of goldenseal constituted less than 1% of the fluorescent signals of DiOC2(3), thus was ignored.

Small interference RNA (siRNA) transfection. Pre-designed siRNAs targeted to human MDR1 (Cat. No. 51320) and a negative control with a scrambled sequence (Cat. No. 4618G) were obtained from Ambion. HepG2 cells seeded in 6 well culture plates were transfected with siRNA using Silencer™ siRNA transfection II Kit (Ambion) following the given instructions. After 3 days, transfected cells were untreated or treated with BBR, CND, or goldenseal for 6 h prior to RNA isolation.

Goldenseal in vivo studies. 42 male Golden Syrian hamsters at 6-8 weeks of age were purchased from the Charles River Laboratories and were housed in cages (3 animals/cage) in an air-conditioned room with a 12 h light cycle. Animals had free access to autoclaved water and food. After one week on a regular rodent chow diet, 36 hamsters were switched to a rodent HFHC diet containing 1.25% cholesterol and 2.2% fat (Product # D12108, Research Diet, Inc., New Brunswick, N.J.) and 6 hamsters were fed a control normal diet containing 0.37% fat and no cholesterol (Product # D12102, Research Diet, Inc.). After 21 days, hamsters on the HFHC diet were randomly divided into 4 groups (n=9 per group) and were given goldenseal at 125 μl/d, 250 μl/d, or BBR 1.8 mg/d by i.p. once a day at 9 AM. The control group received an equal volume of vehicle (20% hydroxypropyl-beta-cyclodextrin (250 μl/animal/d). Goldenseal grain alcohol extract Lot 8 was dried under nitrogen stream and resuspended in 20% hydroxypropyl-beta-cyclodextrin to a final BBR concentration of 7.2 mg/ml. Berberine Chloride was dissolved in the same vehicle solution. Four hours after the last drug treatment, all animals were sacrificed. Blood samples were collected through cardiac puncture and were analyzed for liver function, kidney function, and blood chemistry at DEXX Laboratories (Palo Alto, Calif.). Livers were immediately removed, cut into small pieces, and stored at −80° C. for RNA isolation, protein isolation, and cholesterol content measurement. For histological examination tissues were fixed in OTC under liquid N2 and stored at −80° C. or fixed in 10% paraformaldehyde at room temperature. After staining, tissue sections were evaluated by a veterinary pathologist and an experienced scientist independently. Animal use and experimental procedures were approved by the Institutional Animal Care and Use Committee of the VA Palo Alto Health Care System.

Serum isolation and cholesterol determination. Blood samples (0.2 ml) were collected from the retro-orbital plexus using heparinized capillary tubes under anesthesia (2-3% isoflurane and 1-2 L/min oxygen) after an 8 h fasting (7 AM to 3 PM) before and during the drug treatments. Serum was isolated at room temperature and stored at −80° C. Standard enzymatic methods were used to determine TC, TG, LDL-C, HDL-C and FFA levels with commercially available kits purchased from Stanbio Laboratory (Texas, USA) and Wako Chemical GmbH (Neuss, Germany). Each sample was assayed in duplicate.

Measurement of hepatic cholesterol. 100 mg of frozen liver tissue was thawed and homogenized in 2 ml Chloroform/Methanol (2:1). After homogenization, lipids were further extracted by rocking samples for 1 h at room temperature, followed by centrifugation at 5000 g for 10 min. 1 ml lipid extract was dried under nitrogen stream and redissolved in 1 ml ethanol. TC and free cholesterol were measured using commercially available kits. Cholesterol ester was calculated by subtraction of FC from TC.

HPLC analysis of lipoprotein profiles. Twenty pl of each serum sample from hamsters on a normal diet (n=6), a HFHC diet (n=9), and HFHC diet treated with goldenseal (125 μl/d) (n=9) were pooled. The cholesterol and triglyceride levels of each of the major lipoprotein classes including CM, VLDL, LDL, and HDL in the pool sera were analyzed by HPLC (Okazaki, M et al. Arterioscler. Thromb. Vasc. Biol. 2005, 25: 578-584) at Skylight Biotech, Inc. (Tokyo, Japan).

Western blot analysis of phosphorylated ERK in liver tissues and in HepG2 cells. Approximately 90-100 mg of hamster's liver tissue from each animal was pooled from the same treatment group (n=9) and were homogenized in 5 ml buffer containing 20 mM Tris-HCl pH 8.0, 0.1 M NaCl, 1 mM CaCl₂, cocktails of phosphatase inhibitors (Sigma) and protease inhibitors (complete Mini, Roche Diagnostic). Total homogenate was centrifuged at 800 g for 5 min to pellet nuclei and the supernatant was filtered through muslin cloth. The filtrate was subjected to 100,000 g centrifugation for 1 h at 4° C. to obtain cytosolic fraction. After protein quantitation using BCA™ protein assay reagent (PIERCE), 50 μg protein from each pooled sample was subjected to SDS-PAGE, followed by western blotting using anti-phosphorylated ERK (Cell Signaling) and antibody against total ERK (Santa Cruz). For analyzing ERK activation in HepG2 cells, cells seeded in 6-well culture plates in serum free medium were treated with 10 μg/ml each alkaloid as well as goldenseal (1.5 μl/ml) for 2 h and cell lysates were collected by the method of Kong, W et al. Nature Medicine 2004, 10: 1344-1352.

### Example 1

### Goldenseal Causes Strong Upregulation of LDLR Expression in HEPG2 Cells

Goldenseal contains three major isoquinoline alkaloids BBR, (−)-canadine (CND), and β-hydrastine (HDT), as well as some minor alkaloid components such as hydrastinine (HDTN) (Herbalist, R U American Herbal Pharmacopoeia and Therapeutic Compendium 2001, 1: 1-36; Scazzocchio, F et al. Fitoterapia 1998, 69: 58-59; Weber, H A et al. J. Agric. Food Chem. 2003, 51: 7352-7358) (FIG. 1A). While palmatine (PMT) exists in Coptis, Oregon grape root, and in several other BBR-containing plants (Herbalist, R U 2001 ), only goldenseal contains CND and HDT as native components (Weber, H A et al. J. Agric. Food Chem. 2003, 51: 7352-7358; Weber, H A et al. J AOAC International 2003, 86: 476-483; Betz, J et al. Proceedings of the 39th annual meeting of the American Society of Pharmacognosy 1998, p. 129). Goldenseal root extract typically contains 2.5% to 6% total alkaloids (Weber, H A et al. J AOAC International 2003).

To determine the activity of goldenseal in regulation of LDLR expression, HPLC analysis was first performed on goldenseal ethanol extracts obtained from 8 different commercial suppliers. HPLC/UV-DAD spectroscopic comparisons with standard solutions were used to confirm the presence of BBR, CND, HDT, and HDTN, as well as the absence of PMT. Concentrations of CND and HDT in sample extracts were determined using a single-point calibration and concentrations of BBR in sample extracts were calculated using a standard curve. The identities of BBR, CND, and HDT in extracts were verified further by LC-MS analysis. After these comprehensive quantitative analyses, HepG2 cells were treated for 8 h with goldenseal extract Lot 3 and Lot 6 at a concentration of 2.5 μl/ml (equivalent to a BBR concentration of 15 μg/ml) and with each alkaloid at a concentration of 20 μg/ml. Northern blot analysis showed that HDT, HDTN, and PMT have no effects, but CND and BBR are both strong inducers of LDLR mRNA expression (FIG. 1B). Interestingly, goldenseal extracts with lower BBR concentrations produced the greatest elevation of LDLR mRNA levels. The results of northern blots were independently confirmed by real-time quantitative RT-PCR (FIG. 1C). A 9.8-fold increase in the level of LDLR mRNA was achieved by goldenseal extract Lot 3 that contained 15 μg/ml BBR and 1 μg/ml of CND, whereas BBR at a concentration of 20 μg/ml produced only a 3-fold increase in LDLR mRNA expression. Similar experiments were repeated multiple times using goldenseal extracts from 8 different suppliers. In all assays, goldenseal extracts outperformed the pure compound BBR in the upregulation of LDLR mRNA expression. At comparable concentrations of BBR, the activity of goldenseal extract was typically 2-3 times higher than pure BBR. Goldenseal Lot 8 containing 6.8 μg/μl of BBR and 0.26 μg/μl of CND was thereafter used in all subsequent in vitro and in vivo studies. To confirm the higher potency of goldenseal on LDLR expression, Dil-LDL uptake of HepG2 cells untreated or treated overnight with BBR (10 μg/ml) or goldenseal (1.5 μl/ml) was measured. The LDLR-mediated ligand uptake in HepG2 cells was increased 2.5-fold by BBR and 4.9-fold by goldenseal compared to untreated cells (FIG. 1D).

Previous studies demonstrated that BBR does not activate LDLR gene transcription, but it has a stabilizing effect on LDLR mRNA (Kong, W et al. 2004; Abidi, P et al.Arterioscler. Thromb. Vasc. Biol. 2005, 25: 2170-2176). To determine whether mRNA half-life prolongation is the primary mechanism through which goldenseal elevates LDLR expression, HepG2 cells were transfected with the LDLR promoter luciferase construct pLDLR234Luc along with a normalizing reporter pRL-SV40Luc. After transfection, cells were treated for 8 h with BBR or CND at a concentration of 15 μg/ml, or with 2.2 μ/ml of goldenseal along with two known activators of the LDLR promoter cytokine oncostatin M (OM, 50 ng/ml) (Liu, J et al. J. Biol. Chem. 2000 275: 5214-5221) and the compound GW707 (2 μM) (Grand-Perret, T et al. Nature Medicine 2001, 7: 1332-1338; Liu, J et al. Arterioscler Throm. Vasc. Biol. 2003, 23: 90-96). LDLR promoter activity was strongly elevated by GW707 and OM, but it was not affected at all by goldenseal, CND, or BBR (FIG. 1E). To corroborate this finding further, HepG2 cells were untreated or treated with actinomycin D for 30 min prior to the addition of BBR, CND, or goldenseal, and total RNA was isolated after a 4-h treatment. Real-time quantitative RT-PCR showed that inhibition of transcription by actinomycin D reduced the abundance of LDLR mRNA, but did not prevent the upregulatory effects of these agents on LDLR mRNA expression. Under the same conditions of transcriptional suppression, LDLR mRNA was increased ˜2.5-fold by BBR and CND and 3.4-fold by goldenseal compared to control (FIG. 1F). Collectively, the aforementioned results illustrate that goldenseal extract is highly effective in the upregulation of LDLR expression through mRNA stabilization with a greater activity than the pure compound BBR.

### Example 2

### Increased LDLR Expression by Goldenseal Via the Concerted Synergistic Action of Multiple Bioactive Components in Addition to BBR

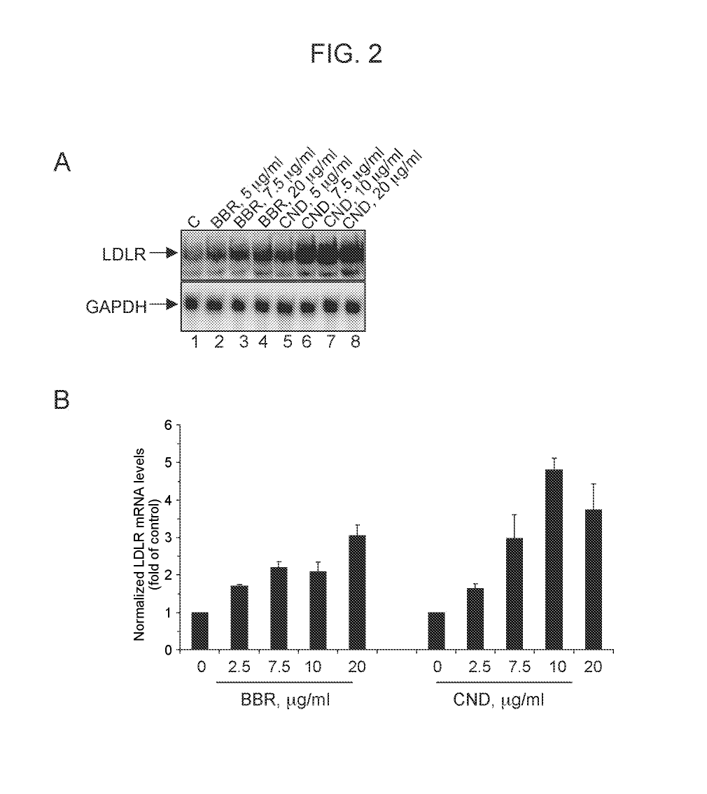

In order to elucidate the molecular mechanisms that confer a potency of goldenseal, a crude BBR-containing mixture, that is higher than the pure compound BBR, the dose-dependent effect of CND with BBR in modulation of LDLR mRNA expression was compared by northern blot analysis (FIG. 2, Panel A) and by quantitative real-time RT-PCR (FIG. 2, Panel B). Within similar concentration ranges, CND increased levels of LDLR mRNA to higher extents than BBR, indicating that CND is a more potent inducer of LDLR expression.

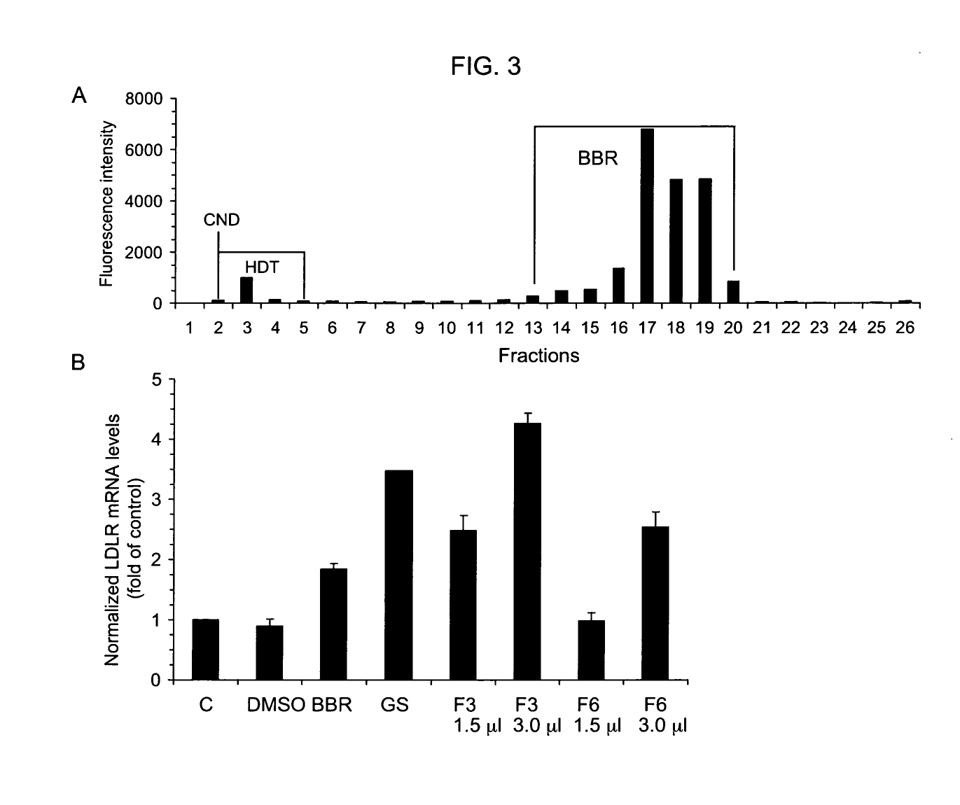

Quantitative HPLC analyses of goldenseal obtained from different suppliers indicated that the amount of CND in goldenseal is significantly lower than BBR, with BBR to CND ratios ranging from 15:1 to 60:1. This implied that CND alone could not account for the 2-3 fold higher activity of goldenseal in the upregulation of LDLR expression. A bioassay driven semi-purification procedure was employed to detect possible LDLR upregulators accompanying BBR and CND in goldenseal. 1 ml of goldenseal ethanol extract was subjected to flash chromatography over a silica gel column with chloroform/methanol in a 10-50% gradient as the eluting solvent, and twenty-six 15 mi-fractions were collected. After evaporation of the solvent, residues in each fraction were dissolved in 250 μl of DMSO and subjected to fluorescence spectroscopy, HPLC, and LC-MS analyses. Based upon the retention time and mass spectrometric characteristics of standard solutions, CND was found in fraction 2; HDT was eluted in fractions 2 to 5; and BBR was identified in fractions 16-20. The majority of the fluorescent material was co-eluted with BBR (FIG. 3, Panel A). Fractions not containing BBR or CND were tested for LDLR modulating activity. HepG2 cells were treated with each fraction at concentrations of 1.5 and 3 μl/ml along with BBR (15 μg/ml) and goldenseal (2.2 μ/ml) for 8 h. The abundance of LDLR mRNA was determined by real-time RT-PCR (FIG. 3, Panel B). The LDLR mRNA level was strongly elevated by fraction 3 (F3) up to 4.3-fold in a dose-dependent manner and was also modestly increased by fraction 6 (F6). The effects of F3 and F6 on pLDLR234Luc promoter activity were tested subsequently. The results showed that similar to BBR and CND, F3 and F6 do not stimulate LDLR transcription (FIG. 1B).

To characterize the components of F3 and F6 further, F3 and F6 were subjected to analysis by HPLC, HPLC-coupled evaporative light scattering detection (ELSD) on a normal phase column, and LC-MS. ELSD detects signal strengths directly proportional to an analyte's mass in the sample (Li, S L et al. J Chromatography A 2001, 909: 207-214), which provides assessments of relative amounts of compounds. The results of these analyses are provided in the figures. FIGS. 13 and 14 are graphs showing the results of ELSD analysis of F3 and F6, respectively. FIGS. 15 and 16 are graphs showing the results of HPLC analysis of F3. FIG. 17 is a graph showing the results of HPLC analysis of F6. FIG. 18 is a graph showing the results of LC-MS analysis of F3. FIG. 19 is a graph showing the results of LC-MS analysis of F6.

ELSD procedure detected 5 single peaks in F3 and the second peak was identified as HDT, which comprised 92% of the mass in F3 (Table 1). Based upon the reference concentration of HDT, concentrations of these compounds in the stock solution ranging from the lowest, 40 μg/ml of F3-5, to the highest, 190 μg/ml of F3-3, were estimated. HPLC-ELSD separated F6 into 5 signal peaks of unknown compounds with estimated concentrations, ranging from 6 μg/ml to 200 μg/ml. FIGS. 13 and 14 are graphs showing the results of ELSD analysis of F3 and F6, respectively.

The results of HPLC analysis of F3 are shown in FIGS. 15 and 16, and results of HPLC analysis of F6 are shown in FIG. 17. Analysis of F6 did not show detectable peaks by HPLC, but peaks were detectable by ELSD analysis. This indicates that F6 may contain sugar moiety(ies) and/or protein moiety(ies). These analyses suggest that F3 contains a canadine-like compound(s), and F6 contains active components structurally different from the berberine-canadine alkaloids.

Because F3 and F6 were added to HepG2 cells at 1:333 dilutions and were able to induce LDLR mRNA expression, the likely effective concentrations of these compounds are estimated to be in the range of 20-600 ng/ml. These data suggest that the compound(s) in F3 and F6 are more potent LDLR modulators than BBR. Taken together, these results indicate that goldenseal increases LDLR expression through a concerted, synergistic action of multiple bioactive compounds in addition to BBR, and that these compounds appear to have greater activities than BBR.

### Example 3

### Significant Attenuation of the Activity of BBR by MDRL Transporter (PGP-170) to Upregulate LDLR Expression in Contrast to Minimal Effect on Goldenseal or CND

A comparison of time-dependent effects of BBR with goldenseal on LDLR mRNA expression revealed that goldenseal elevated the cellular level of LDLR mRNA with .faster kinetics than BBR (FIG. 4, Panel A). To determine whether the difference in kinetics results from different rates of uptake of BBR and its related compounds, HepG2 cells were incubated with 15 μg/ml of BBR, CND, or HDT, or with goldenseal (2.2 μl/ml) for 2 h. Cells were washed with cold PBS and collected through trypsinization. Green fluorescent intensities of BBR in samples were determined by FACS. CND and HDT are not fluorescent and produced only weak background signals similar to untreated control cells. Interestingly, at an equivalent BBR concentration, cells treated with goldenseal had 2.2-fold higher fluorescence than BBR (FIG. 4, Panel B). To examine the kinetics of BBR uptake further, HepG2 cells were incubated with BBR or goldenseal for different times from 0 to 60 min prior to FACS analysis. While the fluorescent intensity increased slowly in a linear fashion in BBR treated cells, it accumulated rapidly in goldenseal treated cells (FIG. 4, Panel C). At 5 min incubation, the fluorescent intensity already increased ˜13-fold in goldenseal-treated cells and increased only ˜2-fold in BBR-treated cells. It is possible that some other minor components of goldenseal are fluorescent and contribute to the higher fluorescent intensity in goldenseal treated HepG2 cells; however, the column separation profile indicated that the majority of the fluorescent signal is derived from BBR (FIG. 3, Panel A).

That the weak antimicrobial action of BBR is caused by an active efflux of BBR from bacteria by multidrug resistance pumps has been reported (Hsieh, P C et al. 1998; Stermitz, F R et al. 2000; Samosorn, S et al. 2006). It is possible that the exclusion of BBR by MDRI transporter (pgp-170) of HepG2 cells is responsible for the low intracellular accumulation thereof. To test this hypothesis, uptakes of BBR and goldenseal for 2 h in HepG2 cells were measured in the absence and the presence of a known MDR1 inhibitor verapamil (VRMP) (Taub, M E et al. Drug Metab. Dispos. 2005, 33: 1679-1687; Stierle, V et al. Biochem. Pharmacol. 2005, 70: 1424-1430) at a dose of 0.6 μM. The green fluorescent intensity in BBR-treated cells was increased significantly by VRMP as demonstrated by direct examination of fluorescent microscopy (FIG. 5A). FACS analysis indicated blocking MDR1 activity with VRMP resulted in a 49% increase in fluorescent intensity in BBR-treated cells but only an 8% increase in goldenseal-treated cells (FIG. 5B). To assess directly the functional role of MDR1 in BBR-mediated LDLR mRNA upregulation, cells were treated with BBR, CND, or goldenseal in the absence or the presence of VRMP and levels of LDLR mRNA were determined. The summarized results from 3 separate experiments showed that VRMP itself had little effect on the LDLR mRNA level, but VRMP produced a 3.6-fold increase in the activity of BBR. In contrast, the activity of goldenseal was increased only marginally (1.3±0.79 fold), and the activity of CND was not affected at all by VRMP (0.82±0.34) (FIG. 5C), suggesting that CND is not a substrate of MDR1.

To examine further the inhibitory role of pgp-170 on BBR activity, HepG2 cells were transfected with siRNA of MDR1 or a control siRNA for 3 days. The transfected cells were treated with BBR for 2 h for measuring BBR uptake or for 6 h for RNA isolation. FACS analysis detected a 49% increase in BBR uptake in MDR1 siRNA transfected cells compared to mock transfected cells (39.54 vs. 26.55). Quantitative RT-PCR showed that the mRNA level of MDR1 was decreased by 69% in control and 71% in BBR treated cells as compared to the nonspecific siRNA transfected cells (mock). Reduction of MDR1 expression by siRNA did not affect LDLR mRNA level in control cells; however, it notably increased the activity of BBR in the elevation of LDLR mRNA level (FIG. 5D). As expected, the activity of CND or goldenseal on LDLR expression was not affected by MDR1 siRNA transfection (data not shown). Altogether, these results clearly demonstrate that MDRI attenuates the activity of BBR on LDLR expression by excluding BBR actively from cells.

The fact that BBR in goldenseal is not excluded by MDRI indicates goldenseal contains a natural MDR inhibitor(s). DiOC2(3), a known fluorescent small molecule, has been widely used as the specific substrate of MDR1 (Minderman, H et al. Cytometry 1996, 25:14-20), and the efflux of DiOC2(3) from cells is inhibited by the nonfluorescent transport substrate vinblastine or the inhibitor VRMP. HepG2 cells were incubated with DiOC2(3) in the absence or the presence of 50 μM VRMP, 15 μg/ml CND, or 2.2 μl/ml goldenseal for 2 h and the retention of DJOC2 was measured by FACS. The efflux of DiOC2(3) was inhibited by goldenseal to a similar degree as by VRMP, whereas CND had no inhibitory effect (FIG. 6, left bar group). In a separate experiment, HepG2 cells were pretreated overnight with vinblastine, VRMP, or goldenseal prior to the addition of DiOC2(3). Again the reduced efflux of DiOC2(3) in goldenseal treated cells was observed, albeit to a lesser extent than with VRMP or vinblastine (FIG. 6, right bar group). Nevertheless, these results, using a known transporter substrate in direct functional assays of MDRI, independently confirmed the finding that goldenseal contains natural MDRI antagonist(s) that accentuate(s) the upregulatory effect of BBR on LDLR mRNA expression.

The MDR inhibitor 5′-methoxyhydnocarpin (5′-MHC) is known to be present in the leaves of Berberis fremontii, a BBR producing plant. However, no peak corresponding to the molecular weight of 5′-MHC was detected in goldenseal. It is likely that the inhibitor(s) produced by goldenseal is(are) structurally different from the one made in Berberis fremontii.

### Example 4

### Eeffective Lowering of Serum Lipid Levels by Goldenseal

To determine whether the strong induction of hepatic LDLR expression renders goldenseal an effective agent in reducing LDL-c from plasma, hyperlipidemic hamsters were used as an animal model to examine the lipid-lowering activity of goldenseal. Thirty-six Golden Syrian male hamsters weighting 110-120 g were fed a high fat and high cholesterol (HFHC) diet for 3 weeks, which significantly increased the fasting serum TC from 137 mg/dl to 549 mg/dl and LDL-c from 76 mg/dl to 364 mg/dl. These animals were divided into 4 treatment groups while they were continuously fed the HFHC diet. One group was treated with BBR at a daily dose of 1.8 mg/animal (15 mg/kg); the second group was treated with goldenseal at a daily dose of 125 μl/animal, equivalent to a BBR dose of 0.9 mg/animal (7.5 mg/kg); the third group was treated with 250 μl of goldenseal per hamster (BBR, 15 mg/kg). The last group received an amount of the 20% hydroxypropyl-beta-cyclodextrin (250 μl/animal/d) vehicle equal to that of the control group. All solutions were administered intraperitoneally (i.p.) once a day for 24 days. Results showed that within the first 10 days of treatment, goldenseal lowered TC, LDL-c, TG, and free fatty acids (FFA) dose-dependently (FIG. 7A-7D). At a half dose of BBR, goldenseal reduced serum lipids to the same levels as BBR. At the same BBR dose, goldenseal produced a more rapid reduction in plasma lipid levels. At the later treatment time points, all drugs reached saturable and steady levels of lipid reduction. The final reductions of serum lipid levels by goldenseal and by BBR as compared to the untreated control group are presented in FIG. 7E. Goldenseal at a daily dose of 125 μl/animal, with an equivalent BBR dose of 0.9 mg/d/animal, reduced plasma TC by 31.3%, LDL-c by 25.1%, TG by 32.6%, and FFA by 44%. This lipid reduction by goldenseal is identical to the lipid lowering effect of BBR at a daily dose of 1.8 mg, thereby demonstrating a two-fold higher potency than BBR in vivo. HPLC analysis of lipoprotein-cholesterol and TG profiles (Okazaki, M et al. 2005) in pooled serum of untreated hamsters on a normal diet, on a HFHC diet, and the low dose goldenseal treated hamsters was performed. HFHC feeding markedly increased the serum levels of VLDL-c, LDL-c, and chylomicron-associated cholesterol in hamsters. Goldenseal treatment reduced cholesterol levels in these lipoproteins without lowering HDL-c (FIG. 7F, upper portion). The TG-lowering effect of goldenseal was also confirmed by the HPLC analysis (FIG. 7F, lower portion).

To correlate directly the LDL-c lowering effects of goldenseal with its ability to upregulate hepatic LDLR expression, at the end of treatment, 6 animals from control and treated groups were sacrificed and levels of liver LDLR mRNA were assessed by quantitative real-time RT-PCR using hamster-specific probes. A 3.2-fold increase by goldenseal (125 ul/d, p<0.0001) and a 3.7-fold increase by BBR (p<0.0001) in LDLR mRNA expression were detected (FIG. 8, Panel A).

Activation of the ERK signaling pathway is a critical event in BBR-mediated upregulation of LDLR expression (Kong, W et al. 2004; Abidi, P et al. 2005). ERK phosphorylation in liver tissues of hamsters was examined. Total cell lysates were prepared from 100 mg of liver tissue and cell lysates from each treatment group (n=9) were pooled. Western blot with anti-phosphorylated ERK demonstrated that levels of phosphorylated ERK were greatly elevated in both goldenseal and BBR treated animals (FIG. 8, Panel B). In addition, ERK activation in HepG2 cells treated with different lots of goldenseal and with individual alkaloids of goldenseal was examined. ERK phosphorylation is induced by goldenseal from different suppliers and this activity is attributable to CND and BBR but not to HDT (FIG. 8, Panel C). Together, these in vivo and in vitro data provide a solid link between modulation of ERK activation and LDLR upregulation by the goldenseal plant.

### Example 5

### Goldenseal Reduces Liver Fat Storage and Inflammation Caused by a High Fat Diet

HFHC feeding increases hepatic cholesterol content and fat storage (Spady, D K et al. J. Clinic. Invest 1988, 81: 300-309; Bensch, W R et al. J. Pharmacol. Experim. Therap. 1999, 289: 85-92). This is often accompanied by inflammation in the liver tissue. To determine whether goldenseal treatment reduces the hepatic fat content in animals fed a HFHC diet, liver tissue sections from animals under different diets and treatment were examined by H&E staining and Oil Red 0 staining. Histological examinations showed that liver tissue from hamsters fed a normal diet displayed a normal lobular architecture with portal areas uniformly approximated. Oil Red O staining showed minimal and scattered lipid staining within small randomly distributed clusters of hepatocytes (FIG. 9, Panel A). In the liver tissues taken from the control HFHC fed hamsters, lipid was massively accumulated in the cytoplasm of hepatocytes as well as inside the portal vein. Furthermore, HFHC diet caused substantial infiltrations of macrophages and mature lymphocytes into the liver tissue (FIG. 9, Panel B). Treatment of hamsters with goldenseal at both doses reduced lipid accumulations in the portal vein and hepatocytes significantly (FIG. 9, Panels C-D). Goldenseal administration also eliminated the inflammatory responses within liver tissue. Restoration of hepatocyte morphology and reduction of liver steatosis were achieved by BBR application as well (FIG. 9, Panel E).

To assess quantitatively the effect of goldenseal in reducing lipid storage, hepatic cholesterol contents in normal fed, HFHC fed control, and HFHC fed and drug-treated hamsters were measured (FIG. 10). As compared to animals fed the normal chow diet, the level of hepatic total cholesterol was increased 6.6-fold (12.5 μmol/g to 82.9 μmol/g) and TG was increased 3.7-fold (18.8 μmol/g to 69.2 μmol/g) in HFHC fed hamsters. These enormous accumulations of cholesterol and TG were markedly reduced in livers of goldenseal-treated animals. Hepatic TC and TG were reduced to 46.5% and 54.3% of control by goldenseal at a daily dose of 125 μ/animal (BBR 0.9 mg/d), whereas BBR at the dose of 1.8 mg/animal/d reduced hepatic TC only to 68.7% and TG to 78.3% of control. These data parallel the results of plasma lipid measurement, further demonstrating that goldenseal extract is extremely effective and exhibits higher potencies than the pure drug BBR in lowering plasma lipid levels and in reducing hepatic accumulations of cholesterol and TG.

### Example 5

### Goldenseal Treatment is Not Associated with Adverse Effects

No adverse effects associated with the drug treatment were observed throughout the entire study. During the 24 day treatment, body weights of animals treated with goldenseal or BBR were unchanged while the body weight of HFHC fed control animals gradually increased by 10% at the end of treatment (FIG. 11, Panel A). Food intake was slightly reduced by the drug treatment (FIG. 11,. Panel B). Compared to HFHC fed control animals, liver function and kidney function were not significantly changed by goldenseal or BBR. The levels of blood glucose were reduced in all treatment groups. HFHC feeding increased the white blood cell (WBC) count by more than 2-fold. Interestingly, this elevation of WBC caused by high fat diet was totally suppressed to the base line level by both doses of goldenseal (p<0.05) and by BBR (p<0.05) (Table 2), indicating that goldenseal has an anti-inflammatory effect.

TABLE 2
Biochemical analyses of hamster blood samples.

| Study group | ALT | AST | ALK | BUN | GLUCOSE | WBC |
|---|---|---|---|---|---|---|
| Normal Diet (n = 4) | 44 ± 10 | 72 ± 44 | 107 ± 16 | 21 ± 2.6 | 143.7 ± 14 | 2866 ± 2371 |
| HFHC, C (n = 5) | 206 ± 65 | 132 ± 20 | 124 ± 22 | 14.6 ± 3.2 | 205 ± 45 | 6450 ± 2585 |
| HFHC, GS 125 ul/day (n = 4) | 229 ± 53 | 182 ± 96 | 99 ± 54 | 19 ± 3.6 | 153.5 ± 46 | 2125 ± 942 * |
| HFHC, GS 250 ul/day (n = 9) | 203 ± 66 | 115 ± 40 | 101 ± 13 | 17.8 ± 1.1 | 123.4 ± 47 * | 2333 ± 1155 * |
| HFHC, BBR 1.8 mg/day | 181 ± 95 | 102 ± 17 | 104 ± 59 | 18.6 ± 5.8 | 152 ± 35 | 2675 ± 1617 * |
| (n = 5) | | | | | | |

* p < 0.05 as compared to HFHC fed control animals.

### Example 6

### Relative Cytotoxicity of CND and BBR

The toxicity of CND and BBR were compared using a cell-based assay. Cell survival rate under drug treatments were determined by Cell Proliferation Kit I (MTT) obtained from Roche Applied Sciences (Indianapolis, Ind.). Cells were seeded in 96-well plates at a density of 5×10³ cells/well/ in 100 μl medium supplemented with 10% FBS at 37° C. and 5% CO₂. After 24 h, cells were incubated with fresh medium containing different concentrations of BBR or CND for 18 h. At the end of drug treatment, 10 μl of MTT labeling reagent per well was added to cells to reach a concentration of 0.5 mg/ml. After a 4 h-reaction, 100 μl of solubilization solution was added to each well and the plate was incubated at 37° C. overnight. The sample spectraphotometrical absorbance was measured by a microplate reader at the wavelength of 550 nm. The reading of sample without drug treatment was defined as 100% survival and readings from drug-treated samples were plotted relative to that value. Quadruple wells were used in each culture condition. Results are shown in FIG. 12.

Any recited method can be carried out in the order of events recited or in any other order that is logically possible. Reference to a singular item includes the possibility that there are plural of the same item present. All patents and other references cited in this application are incorporated into this application by reference except insofar as anything in those patents or references, including definitions, conflicts with anything in the present application (in which case the present application is to prevail).

1. A method of reducing serum lipid in a patient having or suspected of having hyperlipidemia and/or for a medical condition in which lowering serum lipid is beneficial, which comprises administering to said patient:
an effective amount of substantially pure canadine or a pharmaceutically acceptable salt thereof;
an effective amount of one or more substantially pure hypolipidemic and/or hypocholesteremic compounds isolated from the goldenseal plant, or a pharmaceutically acceptable salt of said compound, with the proviso that the compound is not berberine; or
an effective amount of a composition comprising berberine or a pharmaceutically acceptable salt thereof and a multi-drug resistant (MDR) inhibitor or a pharmaceutically acceptable salt thereof.

2. (canceled)

3. The method of claim 1, wherein the substantially pure hypolipidemic and/or hypocholesteremic compounds are isolated from goldenseal root extract.

4.-5. (canceled)

6. A method of raising the HDL-cholesterol:LDL-cholesterol ratio in a patient in need thereof, which comprises administering to said patient:
an effective amount of substantially pure canadine or a pharmaceutically acceptable salt thereof;
an effective amount of one or more substantially pure hypolipidemic and/or hypocholesteremic compounds isolated from the goldenseal plant or a pharmaceutically acceptable salt of said compound, with the proviso that the compound isolated is not berberine; or
an effective amount of a composition comprising berberine or a pharmaceutically acceptable salt thereof and a MDR inhibitor or a pharmaceutically acceptable salt thereof.

7. (canceled)

8. The method of claim 6 claim 7, wherein the substantially pure hypolipidemic and/or hypocholesteremic compounds are isolated from goldenseal root extract.

9.-13. (canceled)

14. The method of claim 1, wherein the one or more substantially pure hypolipidemic and/or hypocholesteremic compounds isolated from the goldenseal plant is selected from:
Factor F3, wherein Factor F3 is produced by isolation from the goldenseal plant by preparative HPLC, and
Factor F6, wherein Factor F6 is produced by isolation from the goldenseal plant by preparative HPLC.

15. (canceled)

16. A method for preventing or treating hyperlipidemia, controlling hyperlipidemia to reduce or prevent cardiovascular disease, preventing or treating one or more symptoms of a cardiovascular disease or condition caused by hyperlipidemia, modulating LDLR expression, and/or modulating ERK activation in a patient in need thereof comprising
administering an anti-hyperlipidemia effective amount of substantially pure canadine or a pharmaceutically acceptable salt, isomer, or enantiomer thereof.

17.-20. (canceled)

21. The method of claim 16, wherein the substantially pure canadine is administered in combination with at least one anti-hyperlipidemic agent or adjunctive therapeutic agent useful in the treatment of cardiovascular disease.

22. A method for increasing LDLR mRNA stability and/or lowering cholesterol in a mammalian cell, tissue, organ, or patient comprising administering to said mammalian cell, tissue, organ, or patient in need of such increasing an effective amount of substantially pure canadine or a pharmaceutically acceptable salt, isomer, or enantiomer thereof.

23.-24. (canceled)

25. A pharmaceutical composition comprising:
i) berberine or a pharmaceutically acceptable salt thereof and ii) an MDR1 multidrug pump inhibitor or a pharmaceutically acceptable salt thereof, wherein i) and ii) are provided in a pharmaceutically acceptable excipient and in separate unit dosage forms;
a mixture of berberine, or a pharmaceutically acceptable salt thereof, and an MDR1 multidrug pump inhibitor, or a pharmaceutically acceptable salt thereof, and a pharmaceutically acceptable excipient;
Factor F3, wherein Factor F3 is produced by isolation from the goldenseal plant by preparative HPLC, and a pharmaceutically acceptable excipient; or Factor F6, wherein Factor F6 is produced by isolation from the goldenseal plant by preparative HPLC, and a pharmaceutically acceptable excipient.

26. A kit comprising the pharmaceutical composition of claim 25, wherein the kit comprises unit doses in separate containers of i)berberine or a pharmaceutically acceptable salt thereof and ii) an MDR 1 multidrug pump inhibitor or a pharmaceutically acceptable salt thereof and an informational and/or instructional package insert.

27.-29. (canceled)

30. A pharmaceutical composition for preventing or alleviating hyperlipidemia in a patient, and/or for increasing LDLR expression and/or increasing LDLR mRNA stabilityin a mammalian cell, tissue, organ, or patient, the pharmaceutical composition comprising:
an anti-hyperlipidemia effective amount of substantially pure canadine or a pharmaceutically acceptable salt, isomer, or enantiomer thereof; and a pharmaceutically acceptable excipient;
an anti-hyperlipidemia effective amount of substantially pure canadine or a pharmaceutically acceptable salt, isomer, or enantiomer thereof, in combination with at least one anti-hyperlipidemic agent or adjunctive therapeutic agent useful in the treatment of cardiovascular disease.

31.-33. (canceled)
